(19) United States
(12) Patent Application Publication
(10) Pub. No.: US 2025/0339464 A1
(43) Pub. Date: Nov. 6, 2025

(54) ENDOGENOUS SIGNALING MOLECULE ACTIVATING CHIMERIC ANTIGEN RECEPTORS AND METHODS OF GENERATION THEREOF

(71) Applicant: MILTENYI BIOTEC B.V. & CO. KG, Bergisch Gladbach (DE)

(72) Inventors: Mira Ebbinghaus, Bergisch Gladbach (DE); Rita Pfeifer, Bergisch Gladbach (DE)

(73) Assignee: MILTENYI BIOTEC B.V. & CO. KG, Bergisch Gladbach (DE)

(21) Appl. No.: 18/864,472

(22) PCT Filed: May 15, 2023
(86) PCT No.: PCT/EP2023/063001

§ 371 Date: Nov. 8, 2024

(30) Foreign Application Priority Data
May 16, 2022 (EP) 22173516.0

(51) Int. Cl. A61K 35/17; A61K 40/11; A61K 40/15; A61K 40/31; A61K 40/42; A61P 35/00; C07K 14/705; C07K 14/715; C07K 14/725; C07K 16/28; G01N 33/569

(52) U.S. Cl. CPC A61K 35/17; A61K 40/11; A61K 40/15; A61K 40/31; A61K 40/421; A61P 35/00; C07K 14/7051; C07K 14/70517; C07K 14/7056; C07K 14/70575; C07K 14/70578; C07K 14/7155; C07K 16/2803; G01N 33/56972; A61K 2239/13; A61K 2239/21; A61K 2239/22

(57) ABSTRACT

The present invention provides an endogenous signaling molecule activating chimeric antigen receptor (ESMA-CAR) comprising a) an antigen binding domain specific for an antigen, b) a first transmembrane domain, and c) an intracellular signaling domain comprising a co-stimulatory domain but no stimulatory domain, wherein said first transmembrane domain, when expressed on the cell surface of an immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR. The present invention also discloses an immune cell expressing said ESMA-CAR and an in-vitro method for the generation of said ESMA-CAR.

### FIELD OF INVENTION

The present invention generally relates to the field of the generation of chimeric antigen receptors (CARs) expressed in immune cells, in particular to the generation of CARs having a specific structure that allow to interact with endogenous signaling molecules in immune cells that allow the activation of said immune cells.

### BACKGROUND OF THE INVENTION

The use of chimeric antigen receptor (CAR)—expressing immune cells such as T cells re-directed to specifically recognize and eliminate malignant cells, greatly increased the scope and potential of adoptive immunotherapy and is being assessed for new standard of care in certain human malignancies. CARs are recombinant receptors that typically target surface molecules in a human leukocyte antigen (HLA)—independent manner. Generally, CARs comprise an extracellular antigen recognition moiety, often a single-chain variable fragment (scFv) derived from antibodies or a Fab fragment, linked to an extracellular spacer, a transmembrane domain and intracellular co-stimulatory and signaling domains. Therapies using CAR-engineered T cells, although sometimes efficacious, have a high potential for improvements, especially with regard to safety.

WO2014055668A1 discloses an immunoresponsive cell comprising a) an antigen recognizing receptor that binds a first antigen with low affinity, wherein binding of the receptor to the first antigen activates the immunoresponsive cell, and b) a chimeric co-stimulating receptor (CCR) that binds a second antigen and stimulates the immunoresponsive cell. Here, two complex recombinant molecules are needed two orchestrate a proper response of the immune cell, wherein one recombinant molecule is the CCR comprising a co-stimulatory domain but no stimulatory domain.

In EP2893004B1 a multi-chain Chimeric Antigen Receptor (CAR) is disclosed, the CAR comprises at least one transmembrane polypeptide comprising at least one extracellular ligand-binding domain, wherein at least one extracellular ligand-binding domain interacts with a cell surface molecule; and one transmembrane polypeptide comprising at least one signal-transducing domain, wherein the signal transducing domain(s) of the multi-chain CAR is present on a polypeptide distinct from that carrying the extracellular ligand-binding domain(s). Regularly at least one transmembrane polypeptide comprises a part of an Fc receptor. Again, here two recombinant molecules are needed to generate a functional CAR that can activate an immune cell.

In WO2014145252A2 activating and inhibiting natural-killer-cell immune-function receptor (NKR) CARs (therein referred to as “NKR-CAR”) are disclosed. An activating NKR-CAR comprises an extra-cellular antigen binding domain; a transmembrane domain and optionally a short NKR cytoplasmic domain. Said NKR-CAR may interact via its transmembrane domain and/or short cytoplasmic domain with an adaptor molecule or intracellular signaling molecule of an immune cell, e.g., a DAP12, FcRy or CD3ζ molecule which can produce an activating signal to said cell. A prominent example in WO2014145252A2 is an activating killer cell immune receptor CAR (actKIR-CAR), namely a KIR2DS2-CAR comprising a KIR2DS2 transmembrane domain and a KIR2DS2 cytoplasmic domain that associates with an adapter molecule having an Immunoreceptor Tyrosine-based Activation Motif (ITAM). Here the concept is that a recombinant receptor without own inherent signaling properties activates an immune cell upon ligand binding via interaction with an endogenous signaling molecule such as DAP12, FcRy or CD3ζ. There is a need in the art for an improved or alternative CAR or CAR concept that may be used for cell immunotherapy.

### BRIEF DESCRIPTION OF THE INVENTION

The inventors developed a novel CAR design that can activate an immune cell that expresses this novel CAR. This CAR is referred to herein as endogenous signaling molecule activating chimeric antigen receptor (ESMA-CAR). This ESMA-CAR has as an intracellular costimulatory domain but no stimulatory domain and a transmembrane domain that can recruit endogenously expressed signaling molecules of the immune cell in which the ESMA-CAR is expressed.

Surprisingly, it was found that despite the missing stimulatory domain, the ESMA-CARs can activate the immune cells in which they are expressed, when the cognate antigens bind to the ESMA-CARs by exploiting endogenous signaling pathways of the immune cells. Normally, costimulatory signals alone are not sufficient to drive the activation of immune cells and require a simultaneous stimulatory signal. In the ESMA-CAR configuration, the CAR can assemble with one or more endogenous stimulatory signaling modules through well-defined transmembrane interactions to build a receptor complex that can potently activate said immune cells. Activation occurs upon antigen binding through the extracellular antigen binding domain leading to structural changes in the CAR transmembrane region and thus also in the transmembrane domains of the endogenous signaling moieties.

The ESMA-CAR has the benefit over the NKR-CAR as disclosed in WO2014145252A2 that the ESMA-CAR mediates a more durable activating signal into the immune cell expressing said ESMA-CAR as disclosed herein compared to an NKR-CAR due to having an own co-stimulatory domain that is not present in activating NKR-CARs.

It was found that the ESMA-CARs exhibit robust in-vitro effector function such as cytotoxicity and cytokine secretion.

Importantly, the cytokine levels produced by ESMA-CAR-expressing immune cells are lower compared to standard single-chain CARs in this way reducing the risk of cytokine release syndrome (CRS) while maintaining potent antitumor cytotoxicity.

Even more surprisingly, it was found that, contrary to the state-of-the-art CARs, the ESMA-CARs displayed high expression of activation markers, but lower upregulation of exhaustion markers on immune cells upon antigen stimulation, the latter allowing for more efficient CAR T cell memory formation, lower activation-induced cell death (AICD) and better persistence of the therapeutic cells in a subject treated with these cells.

A further advantage of the present invention is that the functional ESMA-CAR is a shorter molecule as compared to state-of-the-art CARs that allows e.g. to include additional nucleic acid sequences encoding e.g. for further transgenes such as suicide genes into the vector such as a lentiviral vector, in addition to the nucleic acid encoding the ESMA-CAR.

Surprisingly, not every transmembrane domain of a protein or receptor of an immune cell such as an NK cell that theoretically may be used for expression in an ESMA-CAR configuration as disclosed herein can be expressed as a protein in the immune cell (e.g. transmembrane domains of CD16, CD337, NKG2C, NKG2D and KIR2DS2 do not work as disclosed herein). Therefore, a method for selection of transmembrane domains that may work in ESMA-CAR is an important step for identifying functional ESMA-CARs.

The present invention provides among others said ESMA-CARs, immune cells expressing said ESMA-CARs, a method of creation of an ESMA-CAR, and method for assessing (or determining) the functionality of an ESMA-CAR (for use in immunotherapy).

### BRIEF DESCRIPTION OF THE DRAWINGS

FIG. 1: This is a schematic representation of an ESMA-CAR. The ESMA-CAR is comprised of an antigen-binding domain (here a single-chain variable fragment, scFv), a spacer domain, a transmembrane domain™ and a costimulatory domain. Upon antigen-binding by the scFv, the transmembrane domain recruits an endogenous signaling molecule that contains a stimulatory domain, which initiates a downstream signaling cascade that activates the T cell.

FIG. 2: Expression of different ESMA-CARs on the surface of peripheral blood T cells. (A): CARs that are expressed on the T cell surface. (B): CARs that are not expressed on the T cell surface

FIG. 3: Internalization of the CAR upon ESMA-CAR stimulation to avoid excessive stimulation and activation-induced cell death (AICD)

FIG. 4: activation marker upregulation upon ESMA-CAR stimulation

FIG. 5: exhaustion marker upregulation upon ESMA-CAR stimulation

FIG. 6: longitudinal cytotoxicity of various ESMA-CARs

FIG. 7: cytotoxicity of the ESMA-CARs following stimulation with target cells over an extended period of time

FIG. 8: proliferative capacity of ESMA-CARs following stimulation with target cells over an extended period of time

FIG. 9: a series of graphs depicting the cytokine expression profile of different ESMA-CARs upon antigen stimulation

FIG. 10: T cells expressing the CD335 ESMA-CAR demonstrate effective tumor burden control in vivo.

FIG. 11: Level of exhaustion of human T cells infiltrating the peripheral blood of mice at in vivo study endpoint.

FIG. 12: Blood serum levels of human pro-inflammatory cytokines in mice on day 7 and 14 after systemic treatment with CD335 ESMA CAR T cells, a cognate positive control and untransduced control T cells.

FIG. 13: T cells expressing the CD64 ESMA-CAR demonstrate effective tumor burden control in vivo.

FIG. 14: Surface expression of different ESMA CARs incorporating either a CD28 or 4-1BB costimulatory domain on T cells.

FIG. 15: Upregulation of A) activation and B) exhaustion markers on T cells upon stimulation of CD335 ESMA CARs incorporating either a CD28 or 4-1BB costimulatory domain.

FIG. 16: Secretion of pro-inflammatory cytokines by T cells following antigen-specific stimulation of CD335 ESMA CARs incorporating either a CD28 or 4-1BB costimulatory domain on T cells.

FIG. 17: Time-lapsed cytotoxicity of CD335 ESMA CAR T cells incorporating either a CD28 or 4-1BB costimulatory domain.

FIG. 18: Surface expression of CD335 ESMA CAR in comparison to CD335 TM-intradomain CAR and CD335-intradomainDM-4-1BB ESMA CAR as well as the cognate positive control and untransduced T cells.

FIG. 19: Expression of activation markers on T cells following stimulation of CD335 ESMA CAR, CD335 TM-intradomain CAR, CD335-intradomainDM-4-1BB ESMA CAR as well as the cognate positive control and untransduced T cells

FIG. 20: Secretion of pro-inflammatory cytokines by T cells following antigen-specific stimulation of CD335 ESMA CAR in comparison to CD335 TM-intradomain CAR and CD335-intradomainDM-4-1BB ESMA CAR as well as the cognate positive control and untransduced T cells.

FIG. 21: Longitudinal cytotoxicity of CD335 ESMA CAR in comparison to CD335 TM-intradomain CAR and CD335-intradomainDM-4-1BB ESMA CAR as well as the cognate positive control and untransduced T cells.

### DETAILED DESCRIPTION OF THE INVENTION

In a first aspect the present invention provides an endogenous signaling molecule activating chimeric antigen receptor (ESMA-CAR) comprising or consisting of
a) an antigen binding domain specific for an antigen
b) a first transmembrane domain
c) an intracellular signaling domain comprising a co-stimulatory domain but no stimulatory domain or consisting of a co-stimulatory domain,
wherein said first transmembrane domain, when expressed on (in) the cell surface of an immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

Said ESMA-CAR, wherein said immune cell does not comprise an exogenous (recombinant) protein comprising a stimulatory domain.

Said ESMA-CAR wherein said costimulatory domain of said ESMA-CAR is not able to activate said immune cell without the recruitment of a stimulatory domain of an endogenous signaling molecule of said immune cell.

Said ESMA-CAR, wherein said costimulatory domain of said ESMA-CAR and said first transmembrane domain of said ESMA-CAR may be from different proteins.

Said ESMA-CAR, wherein said first transmembrane domain of said ESMA-CAR does not comprise a cytoplasmic part of the receptor or protein used for said first transmembrane domain.

Said ESMA-CAR, wherein said first transmembrane domain may be from a receptor or protein that does not provide a co-stimulatory signal on its own upon antigen binding.

Said ESMA-CAR, wherein said ESMA-CAR does not comprise a natural killer cell immune-function receptor (NKR) cytoplasmic domain.

Said ESMA-CAR, wherein said ESMA-CAR comprises at least one co-stimulatory domain, e.g. two co-stimulatory domains. Said ESMA-CAR having two co-stimulatory domains may have identical or different co-stimulatory domains.

Said ESMA, wherein said first transmembrane domain may be a transmembrane domain of a receptor naturally expressed in immune cells, but not in said immune cell that expresses said ESMA-CAR.

Said ESMA-CAR, wherein said first transmembrane domain may be a transmembrane domain of a receptor naturally expressed in said immune cell that expresses said ESMA-CAR.

Said immune cell may be e.g. a T cell, e.g. a gamma/delta T cell, a tumor infiltrating lymphocyte, or an NK cell, preferentially said immune cell may be a human immune cell.

Said co-stimulatory domain of the ESMA-CAR may be selected from the co-stimulatory domain of CD27, CD28, 4-1BB (CD137), OX40, CD30, CD40, ICOS, lymphocyte function-associated antigen- 1 (LFA-1), CD2, CD7, LIGHT, 2B4 and DNAM-1.

Said co-stimulatory domain of the ESMA-CAR may comprise a TNF receptor family endodomain, e.g. the co-stimulatory domain of 4-1BB or Ox-40.

Preferentially, said co-stimulatory domain may be 4-1BB.

The co-stimulatory domain of 4-1BB may be or may comprise SEQ ID NO:1.

The co-stimulatory domain of CD28 may be or may comprise SEQ ID NO:2.

Said ESMA-CAR may comprise a hinge. Said hinge may be CD8alpha. Said CD8alpha hinge may have or may comprise the sequence of SEQ ID NO:3.

Said ESMA-CAR, wherein said antigen-binding domain of said ESMA CAR may be an antibody or antigen binding fragment thereof such as a scFv or a nanobody.

Said ESMA-CAR, wherein said ESMA-CAR may comprise two different antigen-binding domains specific for two different antigens or two different epitopes of the same antigen.

Said ESMA-CAR, wherein said first transmembrane domain recruits a stimulatory domain of an endogenous signaling molecule that may be or comprises CD3gamma, CD3delta, CD3epsilon, CD3zeta, DAP10, DAP12 or FcRgamma, or wherein said first transmembrane domain recruits a stimulatory domain of an endogenous signaling molecule that triggers the signaling cascade of CD3gamma, CD3delta, CD3epsilon, CD3zeta, DAP10, DAP12 or FcRgamma.

Said ESMA-CAR, wherein said ESMA-CAR has an at least 40%, at least 50%, at least 60%, at least 70%, at least 80% or at least 90% performance as compared to a reference CAR in an in-vitro assay that allows evaluation (or determination) of functionality and/or efficiency of CARs in immune cells,
wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR.

Such in-vitro assay that allows evaluation (or determination) of functionality and/or efficiency of CARs in immune cells are well-known in the art and also described herein.

Said ESMA-CAR, wherein said ESMA-CAR expressed in said immune cell displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein said at least one cytokine is selected from the group of IL-2, IFN-γ, TNF-α and GM-CSF, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR.

Said ESMA-CAR, wherein said ESMA-CAR expressed in said immune cell displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein said at least one cytokine is selected from the group of IL-2, IFN-γ, TNF-α and GM-CSF, and wherein said ESMA-CAR expressed in said immune cell additionally displays
i) an upregulation of at least one activation marker selected from the group consisting of activation markers CD69, CD25, CD137, and CD154, wherein said upregulation of said at least one activation marker is at least 20-fold, at least 10-fold or at least 5-fold as compared to said immune cell that is not genetically engineered to express a CAR and/or
II) an at least 25%, at least 50% or at least 75% lower upregulation of at least one exhaustion marker as compared to said reference CAR expressed in said immune cell, wherein said at least one exhausting marker is selected from the exhausting markers LAG3, TIM3 and PD-1, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR.

Said reference CAR may have a hinge. Said hinge may be CD8alpha.

The CD3zeta domain may be SEQ ID NO:4.

The transmembrane domain of CD8alpha may be SEQ ID NO:5

The reference CAR exemplary used herein has specificity for EGFR and comprises or has the sequence of SEQ ID NO:6.

Said at least 40%, at least 50%, at least 60%, at least 70%, at least 80% or at least 90% cytotoxicity of said ESMA-CAR expressed in said immune cell against target cells that express said antigen as compared to a reference CAR expressed in said immune cell may be measured by in-vitro longitudinal target cell killing that measures killing of target cells by immune cells that express a CAR specific for an antigen expressed on the surface of said target cells.

Said lower secretion of cytokines may be measured by an in-vitro cytokine secretion assay that provides quantitative analysis of cytokine secretion by CAR immune cells upon antigen recognition.

The in-vitro assays, i.e. the cytokine secretion assay and the longitudinal target cell killing assay and others such as the proliferation capacity assay, the extent of CAR internalization upon antigen engagement assay and the phenotyping assay are assays well-known in the art for assessment of usability of CARs expressed in immune cells. In the following, said in-vitro cytokine secretion assay and the longitudinal target cell killing are described briefly for the general procedure as used herein but without being limited to the exact procedure as described herein as different procedures may exist and may be applicable.

Cytokine secretion assays provide quantitative analysis of cytokine secretion by CAR immune cells such as CAR T cells upon antigen recognition. In this test setting, the MACSPlex bead-based assay technology (Miltenyi Biotec) was used that relies on the same principle as a sandwich ELISA with the minor modification that the capture antibody is bound to soluble beads instead of being immobilized on a plate. The colloidal beads distribute evenly in solution thus enabling a more efficient antigen capturing than the static surface of ELISA well bottoms.

To quantitate the cytokine release of ESMA-CARs, the transgenic immune cells were stimulated with target cells at a ratio 1:2 and incubated for 24 hours. Routinely, co-cultures were set up with 5·10⁴ CAR-positive effector cells and a total volume of 200 μL. Harvested supernatants were analyzed as recommended by the manufacturer (Miltenyi Biotec). Flow cytometric measurements and subsequent data analysis were performed automatically using the MACSQuant® Express Mode for MACSPlex. Measurements of longitudinal target cell killing by CAR immune cells such as CAR T cells was performed using time-lapse imaging. For this, 25,000 GFP-transgenic target cells per well of flat bottomed 96 well plates were seeded and following overnight incubation double the numbers of CAR immune cells such as CAR T cells were added to the cultures. A ratio of 1 to 2 between target and effector cells was used as it reflects the physiological conditions within a tumor. Cultures of target cells only and target cells with mock-transduced immune cells such as T cells were taken along as controls. Phase contrast and green fluorescence images were captured with 10×magnification every two hours for 3-6 days. Analysis of images was performed using the IncuCyte software.

Proliferation capacity assays were conducted by co-culturing ESMA CAR T cells with antigen-positive tumor cells for an extended period of time. On the first day, target cells and CAR+ T cells were seeded at ratio of 1:2. The cultures were routinely setup with 3,2×10⁴ CAR-positive T cells in a volume of 200 μL. Every other day (days 2, 4 and 6), 3,2×10⁴ tumor target cells were added to rechallenge the CAR T cells. On day 1, 3, 5, and 7, samples were taken and the frequency of T cells was quantitated by flow cytometry based on their size and granularity. Cultures of tumor cells only and tumor cells with mock-transduced immune cells such as T cells were taken along as controls.

Extent of CAR internalization upon antigen engagement assay was measured by co-incubating ESMA CAR T cells with antigen-positive target cells. After a 20-24 hour period, CAR T cells were stained extracellularly for CAR expression using a His-tagged EGFR protein that is bound by the scFv and subsequently an anti-His antibody. Samples of CAR T cells without antigen exposure served to assess the baseline CAR expression.

For phenotyping assays, immune CAR cells such as CAR T cell:target cell cocultures were stained for CAR expression as well as with antibodies specific to CD69, CD25, CD3, CD137, CD8, CD4, CD154, PD-1, LAG3, and/or TIM3 according to manufacturers' recommendations.

Said ESMA-CAR, wherein said first transmembrane domain may prevent or impair the homodimerization of the ESMA-CAR and/or may favor the heterodimerization between the first transmembrane domain and the second transmembrane domain.

Said ESMA-CAR, wherein said first transmembrane domain may possess at least one positively charged amino acid.

Said ESMA-CAR, wherein said first transmembrane domain of said ESMA-CAR may be the transmembrane domain of CD64, CD335 or CD336.

Said ESMA-CAR, wherein said first transmembrane domain of said ESMA-CAR may have or may comprise the sequence of SEQ ID NO:7 (CD64), SEQ ID NO:8 (CD335) or SEQ ID NO:9 (CD336).

Other transmembrane domains used herein may have or may comprise the sequence of SEQ ID NO:10 (CD337), SEQ ID NO:11 (CD16), SEQ ID NO:12 (NKG2D), SEQ ID NO:13 (NKG2C), and SEQ ID NO:14 (KIR2DS2).

Preferentially said first transmembrane domain of said ESMA-CAR may be the transmembrane domain of CD335.

Said ESMA-CAR, wherein said first transmembrane domain of said ESMA-CAR may be a transmembrane domain that may interact with an endogenous signaling molecule that comprises CD3zeta.

Said ESMA-CAR, wherein said first transmembrane domain of said ESMA-CAR may be the transmembrane domain of CD335 and wherein said endogenous signaling molecule comprises CD3zeta.

Said ESMA-CAR, wherein said first transmembrane domain of said ESMA-CAR may be the transmembrane domain of CD64 and wherein said endogenous signaling molecule comprises CD3zeta.

Said ESMA-CAR, wherein said first transmembrane domain of said ESMA-CAR may be a transmembrane domain that may interact with an endogenous signaling molecule that comprises DAP12.

Said ESMA-CAR, wherein said first transmembrane domain of said ESMA-CAR may be the transmembrane domain of CD336 and wherein said endogenous signaling molecule comprises DAP12.

Said ESMA-CAR, wherein said first transmembrane domain of said ESMA-CAR may be a transmembrane domain that may interact with an endogenous signaling molecule that comprises FcR gamma.

Said ESMA-CAR, wherein said first transmembrane domain of said ESMA-CAR may be the transmembrane domain of CD64 and wherein said endogenous signaling molecule comprises FcR gamma.

Said antigen may be an antigen expressed on the surface of a target cell such as a cancer cell.

Said antigen may be a soluble antigen, e.g. a soluble antigen that may be coupled to a solid surface or matrix such as a bead, or a soluble antigen that may allow for cross-linking, i.e. that induces dimerization of the CAR.

Exemplary herein the antigen EGFR and an ESMA-CAR having a scFv specific for EGFR (SEQ ID NO:15) was used without the intention to be limited to ESMA-CARs specific for EGFR.

Said ESMA-CAR, wherein said ESMA-CAR may have e.g. an antigen binding domain specific for the antigen MSLN, ROR1, CD318, Fo1R, GD2, TSPAN8, CTLA-4, CD66c, CD276, TEM7, TEM8, PSMA, FAP, CD326, CD90, CD19, CD20 or CD22.

An ESMA-CAR specific for MSLN may have the antigen binding domain of SEQ ID NO:16.

An ESMA-CAR specific for CD318 may have the antigen binding domain of SEQ ID NO:17.

An ESMA-CAR specific for ROR1 may have the antigen binding domain of SEQ ID NO:21.

An ESMA-CAR specific for EGFR may have the antigen binding domain of SEQ ID NO:22.

The ESMA-CAR as disclosed herein may have a CD8alpha hinge, the transmembrane domain of CD335 and the co-stimulatory domain of 4-1BB. Said ESMA-CAR may have regardless of the antigen binding domain the sequence of SEQ ID NO:18.

The ESMA-CAR as disclosed herein may have a CD8alpha hinge, the transmembrane domain of CD336 and the co-stimulatory domain of 4-1BB. Said ESMA-CAR may have regardless of the antigen binding domain the sequence of SEQ ID NO:19.

The ESMA-CAR as disclosed herein may have a CD8alpha hinge, the transmembrane domain of CD64 and the co-stimulatory domain of 4-1BB. Said ESMA-CAR may have regardless of the antigen binding domain the sequence of SEQ ID NO:20.

Said ESMA-CAR, wherein said ESMA-CAR comprises additionally between said first transmembrane domain and said intracellular signaling domain an intracellular stabilizing domain, wherein said intracellular stabilizing domain is from the same receptor or protein as the transmembrane domain, and wherein said intracellular stabilizing domain comprises the complete intracellular domain of said same receptor or protein.

Said ESMA-CAR, wherein said ESMA-CAR comprises additionally between said first transmembrane domain and said intracellular signaling domain an intracellular stabilizing domain, wherein said intracellular stabilizing domain is from the same receptor or protein as the transmembrane domain, and wherein said intracellular stabilizing domain comprises at least 80%, at least 85% at least 90%, at least 95% of the amino acid sequence of the intracellular domain of said same receptor or protein, and wherein the truncation(s) of amino acids of said same receptor or protein is/are from the cytoplasmic end of said same receptor or protein.

Said ESMA-CAR, wherein said ESMA-CAR comprises additionally between said first transmembrane domain and said intracellular signaling domain an intracellular stabilizing domain, wherein said intracellular stabilizing domain is from the same receptor or protein as the transmembrane domain, and wherein said intracellular stabilizing domain comprises between 10 and 70, wherein said amino acids are the amino acids that are intracellularly adjacent of the transmembrane domain of said same receptor or protein.

Said ESMA-CAR, wherein said ESMA-CAR comprises SEQ ID NO:31.

Said ESMA-CAR, wherein said ESMA-CAR comprises SEQ ID NO:32.

Said ESMA-CAR, wherein said ESMA-CAR comprises SEQ ID NO:35.

Said ESMA-CAR, wherein said ESMA-CAR comprises SEQ ID NO:36.

Said ESMA-CAR, wherein said ESMA-CAR comprises SEQ ID NO:37.

Said ESMA-CAR, wherein said ESMA-CAR comprises SEQ ID NO:38.

Said ESMA-CAR, wherein said ESMA-CAR comprises SEQ ID NO:39.

Said ESMA-CAR, wherein said ESMA-CAR comprises SEQ ID NO:40.

Said ESMA-CAR, wherein said ESMA-CAR comprises SEQ ID NO:41.

Said ESMA-CAR, wherein said ESMA-CAR comprises SEQ ID NO:42.

Said ESMA-CAR, wherein said ESMA-CAR may be obtained by the (in-vitro) method (as disclosed also herein) comprising the step of selecting an ESMA-CAR that has a first transmembrane domain that, when expressed on (in) the cell surface of an immune cell, is able to recruit a stimulatory domain of an endogenous signaling molecule of said immune cell, wherein said endogenous signaling molecule is a protein comprising a second transmembrane domain and an intracellular signaling domain comprising said stimulatory domain, and wherein the interaction of the first transmembrane domain and the second transmembrane domain activates said immune cell upon binding of an antigen to said antigen binding domain of said ESMA-CAR.

In one embodiment of the invention said ESMA-CAR is expressed on the cell surface of a T cell, said ESMA-CAR may have preferentially a transmembrane domain selected of transmembrane domains of CD64, CD335 and CD336, and the specificity of the antigen binding domain is for a tumor associated antigen.

In another embodiment of the invention said ESMA-CAR is expressed on the cell surface of an NK cell, said ESMA-CAR may have preferentially a transmembrane domain selected of transmembrane domains of CD64, CD335 and CD336, and the specificity of the antigen binding domain is for a tumor associated antigen.

In another embodiment of the invention said ESMA-CAR is expressed on the cell surface of an tumor infiltrating lymphocyte (TIL), said ESMA-CAR may have preferentially a transmembrane domain selected of transmembrane domains of CD64, CD335 and CD336, and the specificity of the antigen binding domain is for a tumor associated antigen.

In a further aspect the present invention provides an immune cell, e.g. isolated immune cell, expressing an ESMA-CAR, comprising or consisting of
a) an antigen binding domain specific for an antigen
b) a first transmembrane domain
c) an intracellular signaling domain comprising a co-stimulatory domain but no stimulatory domain or consisting of a co-stimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

Said immune cell for use in treatment of a disease in a subject suffering from said disease.

Said disease may be for example cancer, an autoimmune disease or an infectious disease.

Said immune cell, wherein said immune cell may be modified to functionally impair, reduce or eliminate expression of the endogenous T cell receptor (TCR).

Said modification may be achieved e.g. by using gene editing technologies using engineered nucleases that induces a cleavage at a specific cleavage site of a nucleic acid sequence such as a genome of a cell (e.g. TALENs; CRISPR/Cas). Such technologies are well known in the art and e.g. described in WO2018073393.

In a further aspect the present invention provides a nucleic acid, e.g. an isolated nucleic acid, encoding an endogenous signaling molecule activating chimeric antigen receptor (ESMA-CAR) comprising or consisting of
a) an antigen binding domain specific for an antigen
b) a first transmembrane domain
c) an intracellular signaling domain comprising a co-stimulatory domain but no stimulatory domain or consisting of a co-stimulatory domain,
wherein said first transmembrane domain, when expressed on (in) the cell surface of an immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

In a further aspect the present invention provides an immune cell comprising a nucleic acid encoding an ESMA-CAR, comprising or consisting of
a) an antigen binding domain specific for an antigen
b) a first transmembrane domain
c) an intracellular signaling domain comprising a co-stimulatory domain but no stimulatory domain or consisting of a co-stimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

Said nucleic acid may be a lentiviral vector. Said lentiviral vector may comprise additionally a second transgene, e.g. a suicide gene.

In a further aspect the present invention provides an in-vitro method for the creation (generation) of an endogenous signaling molecule activating chimeric antigen receptor (ESMA-CAR) comprising or consisting of
i) an antigen binding domain specific for an antigen
ii) a first transmembrane domain
iii) an intracellular signaling domain comprising a costimulatory domain but no stimulatory domain or consisting of a costimulatory domain,
the method comprising the step of selecting an ESMA-CAR that has a first transmembrane domain that, when expressed on (in) the cell surface of an immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

Said selecting of an ESMA-CAR may comprise the steps of
a) selecting an ESMA-CAR with a first transmembrane domain that can be expressed on (in) the cell surface of an immune cell, and subsequently
b) selecting an ESMA-CAR with said first transmembrane domain that can activate said immune cell upon binding of said antigen to said antigen binding domain of said CAR.

Said first transmembrane domain may be a protein or receptor of an immune cell such as an NK cell that is expressed naturally in said immune cell but is not expressed on (in) the surface of the immune cell that expresses the ESMA-CAR.

Said method, wherein said method comprises identifying an ESMA-CAR of step b) that has an at least 40%, at least 50%, at least 60%, at least 70%, at least 80% or at least 90% performance as compared to a reference CAR in an in-vitro assay that allows evaluation (or determination) of functionality and/or efficiency of CARs in immune cells.

Said method, wherein said method comprises identifying an ESMA-CAR of step b) that, when expressed in said immune cell displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein said at least one cytokine is selected from the group of IL-2, IFN-γ, TNF-α and GM-CSF, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR.

Said method, wherein said method comprises identifying an ESMA-CAR that, when expressed in said immune cell displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein said at least one cytokine is selected from the group of IL-2, IFN-γ, TNF-α and GM-CSF, and wherein said ESMA-CAR, wherein said ESMA-CAR expressed in said immune cell additionally displays
i) an upregulation of at least one activation marker selected from the group consisting of activation markers CD69, CD25, CD137, and CD154, wherein said upregulation of said at least one activation marker is at least 20-fold, at least 10-fold or at least 5-fold as compared to said immune cell that is not genetically engineered to express a CAR and/or
II) an at least 25%, at least 50% or at least 75% lower upregulation of at least one exhaustion marker as compared to said reference CAR expressed in said immune cell, wherein said at least one exhausting marker is selected from the exhausting markers LAG3, TIM3 and PD-1, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR.

In a further aspect the present invention provides an in-vitro method for assessing (or determining) the efficiency (or usability or functionality) of an ESMA-CAR, the method comprising the steps
a) introducing a nucleic acid encoding an ESMA-CAR into an immune cell, said ESMA-CAR comprising or consisting of
i) an antigen binding domain specific for an antigen
ii) a first transmembrane domain
iii) an intracellular signaling domain comprising a costimulatory domain but no stimulatory domain or consists of a co-stimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR,
b) determining if said ESMA-CAR, when expressed in said immune cell, displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein said at least one cytokine is selected from the group of IL-2, IFN-γ, TNF-α and GM-CSF, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR,
and wherein said at least 40%, at least 50%, at least 60%, at least 70%, at least 80% or at least 90% cytotoxicity and said at least 3-fold, at least 5-fold, at least 10-fold, at least 20-fold, at least 30-fold, at least 40-fold lower cytokine secretion of at least one cytokine is indicative for a good efficiency (or usability or functionality) of said ESMA-CAR in said immune cell.

Said-vitro method for assessing (or determining) the efficiency (or usability or functionality) of an ESMA-CAR, the method comprising the steps
a) introducing a nucleic acid encoding an ESMA-CAR into an immune cell, said ESMA-CAR comprising or consisting of
i) an antigen binding domain specific for an antigen
ii) a first transmembrane domain
iii) an intracellular signaling domain comprising a costimulatory domain but no stimulatory domain or consists of a co-stimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR,
b) determining if said ESMA-CAR, when expressed in said immune cell, displays at least 40%, at least 50%, at least 60%, at least 70%, at least 80% or at least 90% cytotoxicity against target cells that express said antigen as compared to a reference CAR expressed in said immune cell and wherein said ESMA-CAR expressed in said immune cell displays upon antigen stimulation an at least 3-fold, at least 5-fold, at least 10-fold, at least 20-fold, at least 30-fold, at least 40-fold lower cytokine secretion of at least one cytokine as compared to said reference CAR expressed in said immune cell, wherein the at least one cytokine is selected from the group of IL-2, IFN-γ, TNF-α and GM-CSF, and additionally determining if said ESMA-CAR, when expressed in said immune cell displays
i) an upregulation of at least one activation marker selected from the group consisting of activation markers CD69, CD25, CD137, and CD154, wherein said upregulation of said at least one activation marker is at least 20-fold, at least 10-fold or at least 5-fold as compared to said immune cell that is not genetically engineered to express a CAR and/or
II) an at least 25%, at least 50% or at least 75% lower upregulation of at least one exhaustion marker as compared to said reference CAR expressed in said immune cell, wherein said at least one exhausting marker is selected from the exhausting markers LAG3, TIM3 and PD-1, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the co-stimulatory domain of said ESMA-CAR,
and wherein said at least 40%, at least 50%, at least 60%, at least 70%, at least 80% or at least 90% cytotoxicity and said at least 3-fold, at least 5-fold, at least 10-fold, at least 20-fold, at least 30-fold, at least 40-fold lower cytokine secretion of at least one cytokine is indicative for a good efficiency (or usability or functionality) of said ESMA-CAR in said immune cell, if additionally said upregulation of said at least one activation marker is at least 20-fold, at least 10-fold or at least 5-fold and/or said upregulation of said at least one exhausting marker is at least 25%, at least 50% or at least 75% lower.

Said in-vitro method for assessing (or determining) the efficiency (or usability or functionality) of an ESMA-CAR, wherein said method comprises after step a) (introducing a nucleic acid encoding an ESMA-CAR into an immune cell) and before step b) (determining the characteristics of the ESMA-CAR), the step of determining if said ESMA-CAR is expressed on (in) the cell surface of said immune cell.

Methods for determining the expression of a recombinant gene on the surface of an immune cell are well-known in the art. Such method may be e.g. the flow cytometric detection of CAR expression by using either a soluble recombinant antigen that is bound specifically by the CAR-scFv, an anti-idiotype antibody that mimics the binding epitope of the cognate antigen or by ProteinL staining. Protein L is a bacterial protein that interacts with the framework regions in the variable domain of immunoglobulin κ light chains and can also to bind to mammalian scFvs.

Said in-vitro method as disclosed herein for assessing (or determining) the efficiency (or usability or functionality) of an ESMA-CAR for use in immunotherapy.

In a further aspect the present invention provides a composition comprising
I) an immune cell expressing an ESMA-CAR, comprising or consisting of
a) an antigen binding domain specific for an antigen
b) a first transmembrane domain
c) an intracellular signaling domain comprising a co-stimulatory domain but no stimulatory domain or consisting of a co-stimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR,
and
b) a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Said composition for use in treatment of a disease (in a subject) such as cancer, an infectious disease or an autoimmune disease.

In a further aspect the present invention provides in in-vivo method for treatment of a subject suffering from a disease, the method comprising administering to said subject an immune cell expressing an ESMA-CAR, comprising or consisting of
a) an antigen binding domain specific for an antigen
b) a first transmembrane domain
c) an intracellular signaling domain comprising a co-stimulatory domain but no stimulatory domain or consisting of a costimulatory domain,
wherein said first transmembrane domain, when expressed on (in) the cell surface of said immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

Said disease may be cancer, an infectious disease or an autoimmune disease.

In one embodiment of the invention the immune cells expressing the ESMA-CAR as disclosed herein are for use in treatment of a disease associated with a target cell of a subject suffering from said disease, the disease may be e.g. cancer and the target cell a cancerous cell. Immune cells, e.g. T cells or NK cells of a subject may be isolated. The subject may e.g. suffer from said cancer or may be a healthy subject. These cells are genetically modified in vitro to express the ESMA-CAR as disclosed herein. These engineered cells may be activated and expanded in vitro. In a cellular therapy these engineered cells are infused to a recipient in need thereof. These cells may be a pharmaceutical composition (said cell plus pharmaceutical acceptable carrier). The infused cells may be e.g. able to kill (or at least stop growth of) cancerous cells in the recipient. The recipient may be the same subject from which the cells was obtained (autologous cell therapy) or may be from another subject of the same species (allogeneic cell therapy).

The immune cells, preferentially T cells or NK cells engineered to express the ESMA-CAR as disclosed herein may be administered either alone, or as a pharmaceutical composition in combination with diluents and/or with other components such as IL-2 or other cytokines or cell populations. Briefly, pharmaceutical compositions of the present invention may comprise a cell population of genetically modified cells (a plurality of immune cells) as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Preferentially, the compositions of the present invention are formulated for intravenous administration. The administration of cell compositions to the subject may be carried out in any convenient manner known in the art.

Pharmaceutical compositions of the present invention may be administered in a manner appropriate to the disease to be treated. Appropriate dosages may be determined by clinical trials. But the quantity and frequency of administration will also be determined and influenced by such factors as the condition of the patient, and the type and severity of the patient's disease.

A pharmaceutical composition comprising the immune cells, preferentially T cells or NK cells as disclosed herein may be administered at a dosage of 10⁴ to 10⁹ cells/kg body weight, preferably 10⁵ to 10⁶ cells/kg body weight. The cell compositions may also be administered several times at these dosages. The compositions of cells may be injected e.g. directly into a tumor, lymph node, or site of infection.

The genetically engineered immune cells may be activated and expanded to therapeutic effective amounts using methods known in the art.

The immune cells of the invention may be used in combination with e.g. chemotherapy, radiation, immunosuppressive agents, antibodies or antibody therapies.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

In general, a “standard” CAR may comprise an extracellular domain (extracellular part) comprising the antigen binding domain, a transmembrane domain and a cytoplasmic signaling domain (intracellular signaling domain). The extracellular domain may be linked to the transmembrane domain by a linker (or spacer or hinge or hinge region). The extracellular domain may also comprise a signal peptide.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies, nanobodies (or VHH or single domain antibody) or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain).

The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, ICOS, lymphocyte function-associated antigen- 1 (LFA-1), CD2, CD7, LIGHT.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

The CARs may be designed to comprise any portion or part of the above-mentioned domains as described herein in any order and/or combination resulting in a functional CAR, i.e. a CAR that mediated an immune effector response of the immune effector cell that expresses the CAR.

The term “ESMA-CAR” as used herein refers to a CAR comprising two parts. One part is the recombinant part of the ESMA-CAR and comprises the antigen binding domain specific for an antigen, a transmembrane domain and a co-stimulatory domain but no stimulatory domain as disclosed herein. The first part may also comprise optionally a hinge region between the antigen binding domain and the transmembrane domain. This first part is similar to the “standard” CAR described above with the difference that the cytoplasmic signaling domain does not comprise a stimulatory domain and the transmembrane domain has specific features not common to “standard” CARs as described herein.

The second part of the ESMA-CAR is an endogenous signaling molecule of said immune cell. Said endogenous signaling molecule of said immune cell is a protein comprising a transmembrane domain and an intracellular signaling domain comprising a stimulatory domain.

Both parts need to interact for enabling the activation of said immune cell, when an antigen binds to the antigen binding domain of said ESMA-CAR. The recombinant first part of the ESMA-CAR is not sufficient to activate said immune cell without interaction with the second, the endogenous part of the ESMA-CAR, i.e. if no interaction between the first part and the second part occurs, no activation of the immune cell occurs.

The transmembrane domain of the recombinant part (=first transmembrane domain) and the transmembrane domain of the second part can interact with each other and activate jointly said immune cell, when an antigen binds to the antigen-binding domain of the ESMA-CAR. In other words, the first part of the ESMA-CAR recruits via its transmembrane domain the second part of the ESMA-CAR by allowing interaction of the first transmembrane domain with the second transmembrane domain.

Said first transmembrane domain may be a transmembrane domain of a receptor naturally expressed in immune cells, preferentially in other types of immune cells but may be not expressed naturally in said immune cell that expresses the ESMA-CAR.

Said first transmembrane domain may prevent or may impair the homodimerization of the ESMA-CAR and/or may favor the heterodimerization between the first transmembrane domain and the second transmembrane domain.

Said impairment of homodimerization may be at least 40%, at least 50%, at least 60%, at least 70%, at least 80%, at least 90% as compared to the homodimerization that occurs when a reference CAR having CD8alpha as transmembrane domain is used.

Said first transmembrane domain possesses at least one positively charged amino acid.

Said co-stimulatory domain of the ESMA-CAR may be CD27, CD28, 4-1BB (CD137), OX40, CD30, CD40, ICOS, lymphocyte function-associated antigen- 1 (LFA-1), CD2, CD7, LIGHT, or NKG2C, B7-H3.

Preferentially, said co-stimulatory domain may be 4-1BB.

Said ESMA-CAR may comprise a hinge. Said hinge may be CD8alpha.

The antigen-binding domain of the ESMA-CAR may be an antibody or antigen binding fragment thereof such as a scFv or a nanobody.

Said ESMA-CAR may recruit a stimulatory domain of an endogenous signaling molecule that is or triggers the signaling cascade of CD3zeta, CD3gamma, CD3epsilon, CD3delta, DAP10, DAP12 or FcRgamma.

The term “intracellular stabilizing domain” or “intracellular domain” of an ESMA-CAR as used herein may be used interchangeably and refer to a domain or amino acid sequence that follows directly in a receptor or protein to the domain or amino acid sequence that build the transmembrane domain of said receptor or protein and reaches into the intracellular part of a cell in which the receptor or protein is expressed.

The intracellular stabilizing domain may comprise the complete intracellular domain of said same receptor or protein, or may comprise at least 80%, at least 85% at least 90%, at least 95% of the amino acid sequence of the intracellular domain of said same receptor or protein, and wherein the truncation(s) of amino acids of said same receptor or protein is/are from the cytoplasmic end of said same receptor or protein.

The intracellular stabilizing domain may comprise between 10 and 70 amino acids.

The presence of said intracellular stabilizing domain in an ESMA-CAR may further enhance the efficiency of the ESMA-CAR. Without being bound to this theory, this is thanks to further interaction of the ESMA-CAR with the endogenous signaling molecule that interacts with the ESMA-CAR as disclosed herein, thereby stabilizing the interaction of the recombinant and exogenous ESMA-CAR part with said endogenous signaling molecule.

The term “activates the immune cell” in the context of activation of an ESMA-CAR means an induction of a signaling cascade which is associated with altered gene expression status in the immune cell initiating an immune response which includes, but is not limited to, proliferation, differentiation, cytokine release, cytolytic effector function and the like. The term “mediates an immune response of the immune cell” may have the same meaning as “activates the immune cell” and may be used interchangeably.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”). Examples of antigen binding fragments include Fab (fragment antigen binding), scFv (single chain fragment variable), single domain antibodies or VHH or nanobodies, diabodies, dsFv, Fab′, diabodies, single-chain antibody molecules, and multispecific antibodies formed from antibody fragments.

The terms “having specificity for”, “specifically binds” or “specific for” with respect to an antigen-binding domain of an antibody, of a fragment thereof or of a ESMA-CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The cluster of differentiation (CD) is a protocol used for the identification and investigation of cell surface molecules providing targets for immunophenotyping of cells. In terms of physiology, CD molecules can act in numerous ways, often acting as receptors or ligands important to the cell. A signal cascade is usually initiated, altering the behavior of the cell.

As used herein “autologous” means that cells, a cell line, or population of cells used for treating subjects are originating from said subject.

As used herein “allogeneic” means that cells or population of cells used for treating subjects are not originating from said subject but from a donor.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, Th9, or TFH, which secrete different cytokines to facilitate a different type of immune response. Signaling from the APC directs T cells into particular subtypes.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

$$Two major classes of CD 4 + Treg cells have been described - Foxp 3 + Treg cells and Foxp 3 - Treg cells .$$

Natural killer T cells (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CDld. Once activated, these cells can perform functions ascribed to both Th and Tc cells (i.e., cytokine production and release of cytolytic/cell killing molecules).

The term “natural killer cells (NK cells)” are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8. Continuously growing NK cell lines can be established from cancer patients and common NK cell lines are for instance NK-92, NKL and YTS. The terms “immune cell” or “immune effector cell” may be used interchangeably and refer to a cell that may be part of the immune system and executes a particular effector function such as alpha-beta T cells, NK cells, NKT cells, B cells, innate lymphoid cells (ILC), cytokine induced killer (CIK) cells, lymphokine activated killer (LAK) cells, gamma-delta T cells, monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells. Most preferred immune effector cells are T cells and NK cells. “Effector function” means a specialized function of a cell, e.g. in a T cell an effector function may be cytolytic activity or helper activity including the secretion of cytokines.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “epitope” means the part of an antigen, e.g. a soluble antigen, that may be recognized and specifically bound by antibodies or antigen bindings fragments thereof (antigen binding domains).

The tumor associated antigen (TAA) as used herein refers to an antigenic substance produced in tumor cells. Tumor associated antigens are useful tumor or cancer markers in identifying tumor/cancer cells with diagnostic tests and are potential candidates for use in cancer therapy. Preferentially, the TAA may be expressed on the cell surface of the tumor/cancer cell, so that it may be recognized by the antigen binding receptor as disclosed herein.

The term “target cell” as used herein refers to cell which expresses an antigen on its cell surface that should be recognized (bound) by the antigen binding domain of the ESMA-CAR as disclosed herein or by the antigen binding domain of the tag of the tagged polypeptide as disclosed herein.

Said target cell may be e.g. a cancerous cell or a cell associated with an autoimmune disease or a cell associated with an infectious disease.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR immunotherapy” or in case of use of T cells only as “CAR T cell therapy” or “CAR T cell immunotherapy”.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the individual is a human. The subject may be a subject suffering from a disease such as cancer.

The term “expression” as used herein is defined as the transcription and/or translation of a particular nucleotide sequence driven by its promoter in a cell.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The term “expression” as used herein is defined as the transcription and/or translation of a particular nucleotide sequence driven by its promoter in a cell.

The term “introducing a nucleic acid sequence into a cell” means that nucleic acids such as DNA and/or RNA are introduced into a cell by methods well-known in the art for allowing the cell to uptake nucleic acids. Such methods are e.g. transfection, transduction, magnetofection and electroporation.

The term “isolated” is used herein to indicate that the polypeptide, nucleic acid or host cell exist in a physical milieu distinct from that in which it occurs in nature. For example, the isolated polypeptide may be substantially isolated (for example enriched or purified) with respect to the complex cellular milieu in which it naturally occurs, such as in a crude extract.

### EXAMPLES

The following examples are intended for a more detailed explanation of the invention but without restricting the invention to these examples.

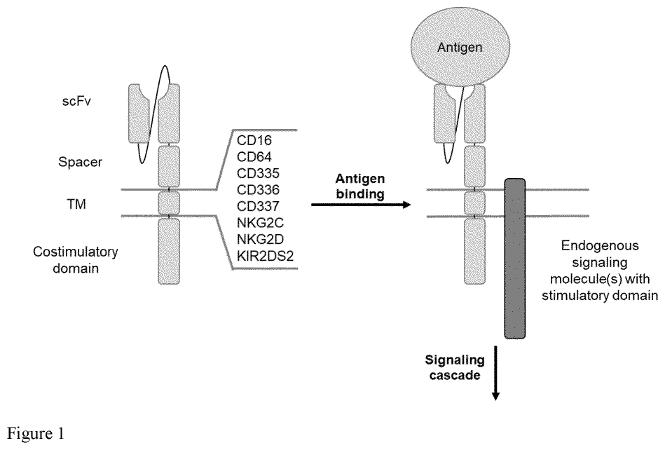

### Example 1: Design of Different ESMA-CARs as Shown in FIG. 1

Multiple ESMA-CARs were created by integration of different transmembrane domains into a CAR targeting the EGFR. Apart from the transmembrane domain, the CARs contain an scFv that targets the EGFR, a CD8α hinge domain and a costimulatory 4-1BB domain. A CAR with the structure anti-EGFR-scFv_CD8α-hinge_CD335-TM_4-1BB was created, as detailed in SEQ ID NO:23. A second CAR has the structure anti-EGFR-scFv_CD8α-hinge_CD336-TM_4-1BB, as detailed in SEQ ID NO:24. A third CAR with the structure anti-EGFR-scFv_CD8α-hinge_CD64-TM_4-1BB is detailed in SEQ ID NO:25. Additionally, CARs containing the CD16, CD337, NKG2D, NKG2C or KIR2DS2 transmembrane domain were created, which were not expressed on the T cell surface. The CARs have the structure anti-EGFR-scFv_CD8α-hinge_TM_4-1BB, as detailed in SEQ ID:26 (CD16 TM), SEQ ID:27 (CD337 TM), SEQ ID:28 (NKG2D TM), SEQ ID:29 (NKG2C TM) and SEQ ID:30 (KIR2DS2 TM).

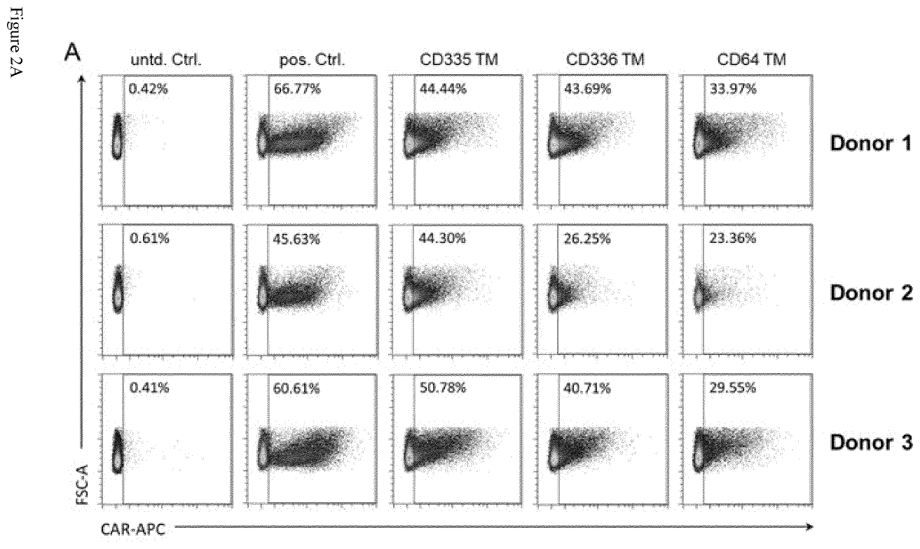

### Example 2: Expression of Different ESMA-CARs on the Surface of Peripheral Blood T Cells, as Shown in FIG. 2

Peripheral blood mononuclear cells (PBMCs) were isolated from buffy coats using gradient centrifugation with Pancoll, human (Pan Biotech). T cells were isolated from PBMCs using the Pan T cell Isolation Kit, human (Miltenyi Biotec). T cells were activated in TexMACS™ medium (Miltenyi Biotec) supplemented with TransAct™ reagent (Miltenyi Biotec), 100 IU/mL of recombinant human IL-2 IS, research grade (Miltenyi Biotec) and 100 μg/mL Primocin® (InvivoGen). Twenty-four hours after isolation, the activated T cells were transduced with CAR-encoding VSV-G pseudo typed lentiviral vectors. Forty-eight hours post transduction, TransAct™ was removed from the cells by medium exchange to TexMACS™ medium supplemented with IL-2 and Primocin®. Cells were expanded for 14 days and CAR expression levels were analyzed on day 14 by flow cytometry.

Surface expression was determined with flow cytometry using an EGFR protein with His-tag (Acrobiosystems) that is bound by the CAR scFv and detected by secondary anti-His antibody (APC or PE, Miltenyi Biotec). Expression of a positive control CAR (pos. Ctrl.) on the surface of T cells and non-modified T cells (untd. Ctrl.) are shown for comparison. CAR expression on at least 20% of the T cells was detectable for the CD335, CD336 and CD64 transmembrane encompassing ESMA-CARs (FIG. 2A), whereas no expression was detectable for the CD16, CD337, NKG2D, NKG2C and KIR2DS2 transmembrane-containing CARs (FIG. 2B). As there was no expression of the CD16, CD337, NKG2D, NKG2C and KIR2DS2 transmembrane-containing CARs on the T cell surface, the CD335, CD336 and CD64 transmembrane containing ESMA-CARs were used in different assays.

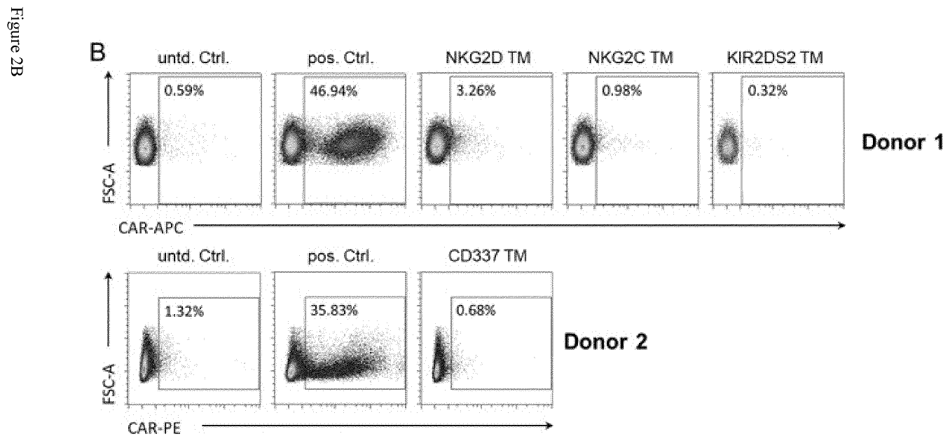

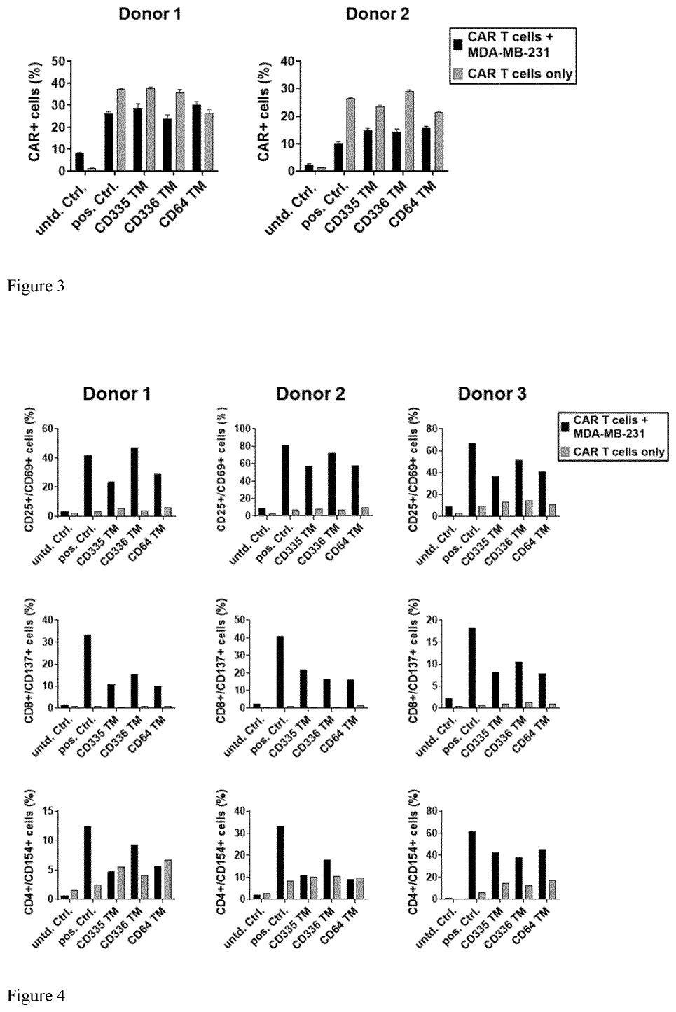

### Example 3: Internalization of the CAR Upon ESMA-CAR Stimulation (Depicted in FIG. 3)

5*10⁴ CAR+ T cells were co-cultured with tumor target cells (MDA-MB-231 cells) at 1:2 ratio in DMEM (Biowest) with 10% FBS (Catus Biotech) and Primocin® (InvivoGen) for 20 to 24 hours. Then, CAR surface expression was determined with flow cytometry measurement using an EGFR protein with His-tag that is bound by the CAR scFv and detected by secondary anti-His antibody (APC, Miltenyi Biotec). In addition, CAR+ T cells were cultured without target cells to determine the CAR baseline expression rate for each ESMA-CAR. CAR surface expression of positive control CAR T cells (pos. Ctrl.) co-cultured with and without target cells was also determined as comparison. To estimate the background signal of the EGFR-His protein and anti-His antibody, unmodified T cells were cultured with and without target cells, stained and detected with flow cytometry. CAR expression on the T cell surface was diminished after stimulation of the ESMA-CARs and positive control CAR except for the CD64 transmembrane-containing CAR in one experiment (Donor 1). The experiment was performed with two donors in triplicate. Mean CAR expression and standard deviation are indicated for each sample.

### Example 4: Activation Marker Upregulation Upon ESMA-CAR Stimulation, Graphs Shown in FIG. 4

5*10⁴ CAR+ T cells were co-cultured with tumor target cells (MDA-MB-231 cells) at 1:2 ratio in DMEM (Biowest) with 10% FBS (Catus Biotech) and Primocin® (InvivoGen) for 20 to 24 hours. Then, expression of different activation markers was determined with flow cytometry measurement. CAR expression was determined with EGFR protein with His-tag and secondary anti-His antibody (PE) and expression of the activation markers was determined with fluorescently-labeled antibodies against each marker (Miltenyi Biotec). Additionally, CAR+ T cells were cultured without target cells to determine background expression of the activation markers. As a control, unmodified T cells (untd. Ctrl.) were cultured with and without target cells to determine unspecific activation of the T cells by the target cells. First, CAR+ T cells were identified flow cytometrically, then the expression of each activation marker on the CAR+ T cells was quantified.

Upon stimulation of the ESMA-CARs with tumor target cells, activation markers CD25 and CD69 were upregulated in CAR T cells at comparable or lower level to positive control (pos. Ctrl.) CAR T cells. CD137 was upregulated in CD8+ ESMA-CAR T cells at lower level than the positive control CAR. CD154 was only upregulated in CD4+ CAR T cells in some of the experiments and for some of the ESMA-CARs.

The experiment was performed with three donors. The percentage of CAR+ T cells that express each activation marker is depicted. For the unmodified control T cells, the percentage of T cells (determined by size and granularity in flow cytometry) that express each activation marker is depicted. CAR expression was detected in all CAR T cells. CD25 and CD69 expression was detected in duplicate, the mean is shown. CD4, CD8, CD154 and CD137 expression was measured in singlets.

### Example 5: Exhaustion Marker Upregulation Upon ESMA-CAR Stimulation (FIG. 5)

5*104 CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. Then, expression of different exhaustion markers was determined with flow cytometry measurement. CAR expression was determined with EGFR protein with His-tag and secondary anti-His antibody (PE) and expression of the exhaustion markers was determined with fluorescently-labeled antibodies against each marker (Miltenyi Biotec). Additionally, CAR+ T cells were cultured without target cells to determine background expression of the activation markers. As a control, unmodified T cells (untd. Ctrl.) were cultured with and without target cells to determine unspecific activation of the T cells by the target cells that could lead to exhaustion of the T cells. First, CAR+ T cells were identified flow cytometrically, then the expression of exhaustion markers was quantified sequentially. First, PD1+ CAR T cells were identified, followed by quantification of Lag3 and Tim3 expression on the PD1+/CAR+ T cells. Upon stimulation of the ESMA-CARs with tumor target cells, a combined expression of the exhaustion markers PD1, Lag3 and Tim3 was observed at comparable or lower level to positive control (pos. Ctrl.) CAR T cells. The CD336 transmembrane-containing ESMA-CAR T cells showed comparable combined expression of the three exhaustion markers in two out of three donors (donor 1 and 3), in the third donor (donor 2) the expression was lower than in the pos. Ctrl. CAR T cells. The CD335 and CD64 transmembrane-containing ESMA-CAR T cells expressed lower levels of the three exhaustion markers compared to the pos. Ctrl. CAR T cells. The experiment was performed with three donors. The percentage of CAR+ T cells that express all three exhaustion markers is depicted. For the unmodified control T cells, the percentage of T cells (determined by size and granularity in flow cytometry) that express all three exhaustion markers is depicted. CAR expression as well as PD1, Tim3 and Lag3 expression was measured in singlets.

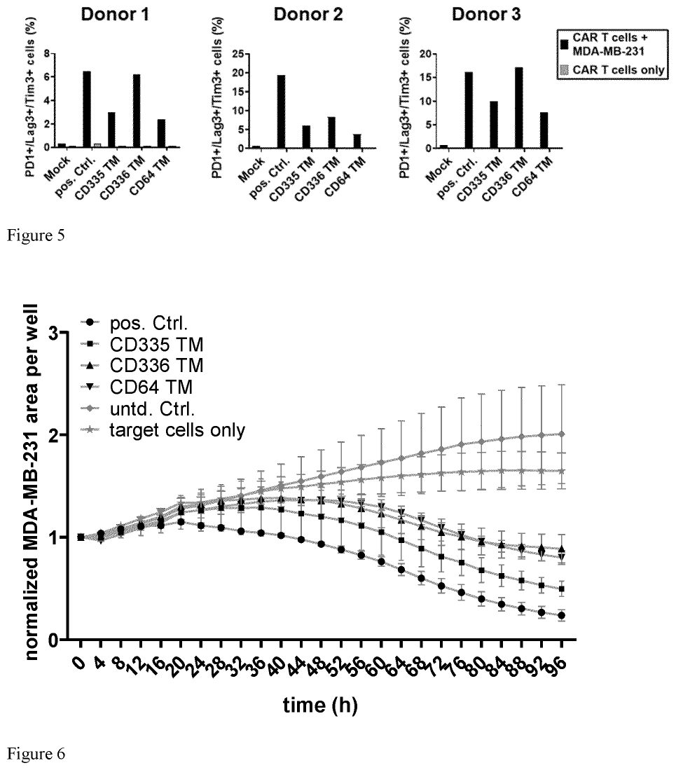

### Example 6: Longitudinal Cytotoxicity of Various ESMA-CARs, Depicted in FIG. 6

5*10⁴ CAR+ T cells were co-cultured with GFP+/Luciferase+ tumor target cells (MDA-MB-231 cells) at 2:1 ratio in DMEM (Biowest) with 10% FBS (Catus Biotech) and Primocin® (InvivoGen) for four days. To determine cytotoxic efficiency of the CAR+ T cells against the tumor cells, the growth of tumor cells was analyzed with an IncuCyte® S3 Life-Cell Analysis System (Sartorius). The green surface area of the target cells was measured over time and values normalized to the area at the start of the experiment (0h). To determine tumor cell growth without cytotoxic effects of the T cells, target cells were cultured without T cells as a control (target cells only). Target cell growth in co-culture with positive control (pos. Ctrl.) CAR T cells and unmodified T cells (untd. Ctrl.) were measured as a positive and negative control, respectively.

All ESMA-CARs could diminish tumor cell growth, albeit at lower level than the pos. Ctrl. CAR T cells. The CD335 transmembrane domain-containing ESMA-CAR could decrease target cell numbers whereas the CD336 and CD64 transmembrane domain-containing ESMA-CARs could control tumor growth. The experiment was performed with three donors in triplicate, representative data is shown.

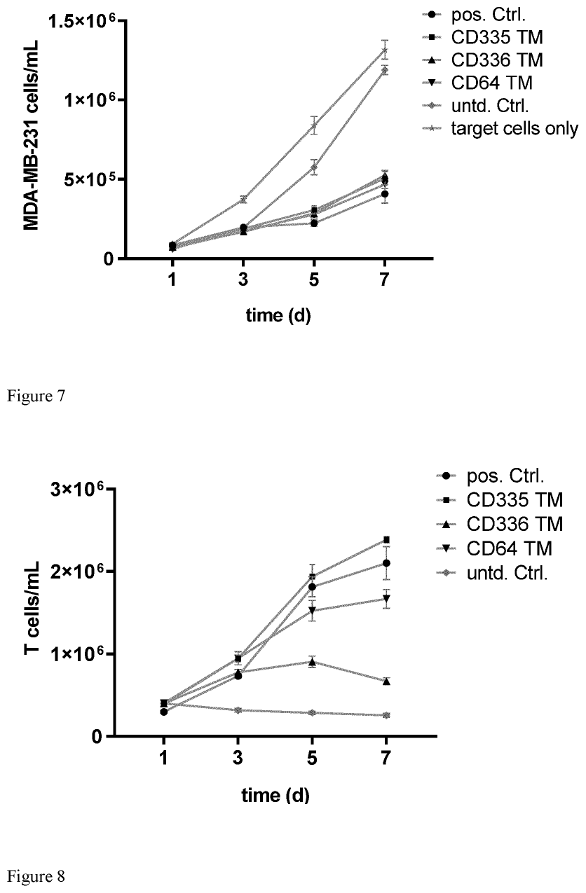

### Example 7: Cytotoxicity of the ESMA-CARs Following Stimulation with Target Cells Over an Extended Period of Time, Graph Shown in FIG. 7

1.6*10⁴ GFP+/Luciferase+ tumor target cells (MDA-MB-231 cells) were plated on four 96 well plates and co-cultured with 3.2*10⁴ CAR+ T cells in DMEM (Biowest) with 10% FBS (Catus Biotech) and Primocin® (InvivoGen). To determine tumor cell growth without cytotoxic effects of the T cells, target cells were cultured without T cells as a control (target cells only). Target cell growth in co-culture with positive control CAR T cells (pos. Ctrl.) and unmodified T cells (untd. Ctrl.) were measured as a positive and negative control, respectively. After 1, 3, 5 and 7 days, one plate was removed and GFP+ target cells counted with flow cytometry. For the remaining plates, 100 μL supernatant were removed and 3.2*10⁴ target cells added at day 2, 4, and 6.

All ESMA-CARs could diminish tumor cell growth at comparable level to the pos. Ctrl. CAR over an extended period of seven days. The experiment was performed with two donors in triplicate, representative data is shown. Mean GFP+ target cell count per mL and standard deviation are indicated.

### Example 8: FIG. 8 Depicts the Proliferative Capacity of ESMA-CARs Following Stimulation with Target Cells Over an Extended Period of Time

1.6*10⁴ GFP+/Luc+ tumor target cells (MDA-MB-231 cells) were plated on four 96 well plates and co-cultured with 3.2*10⁴ CAR+ T cells in DMEM (Biowest) with 10% FBS (Catus Biotech) and Primocin® (InvivoGen). Positive control CAR T cells (pos. Ctrl.) and unmodified T cells (untd. Ctrl.) were measured as a positive and negative control, respectively. After 1, 3, 5 and 7 days, one plate was removed and T cells counted with flow cytometry. T cells were identified by size and granularity in the flow cytometry measurement. For the remaining plates, 100 μL supernatant were removed and 3.2*10⁴ target cells added at day 2, 4, and 6. All ESMA-CARs proliferated after stimulation with target cells. The CD335 transmembrane domain-containing ESMA-CAR T cells proliferated at higher level than the positive control CAR T cells, whereas the CD64 transmembrane domain-containing CAR T cells proliferated at slightly lower level. The CD336 transmembrane domain-containing CAR T cells proliferated at lowest level and T cell count decreased after day five. The untransduced Ctrl. T cells did not proliferate and cell counts decreased over time. The experiment was performed with two donors in triplicate, representative data is shown. Mean T cell count per mL and standard deviation are indicated.

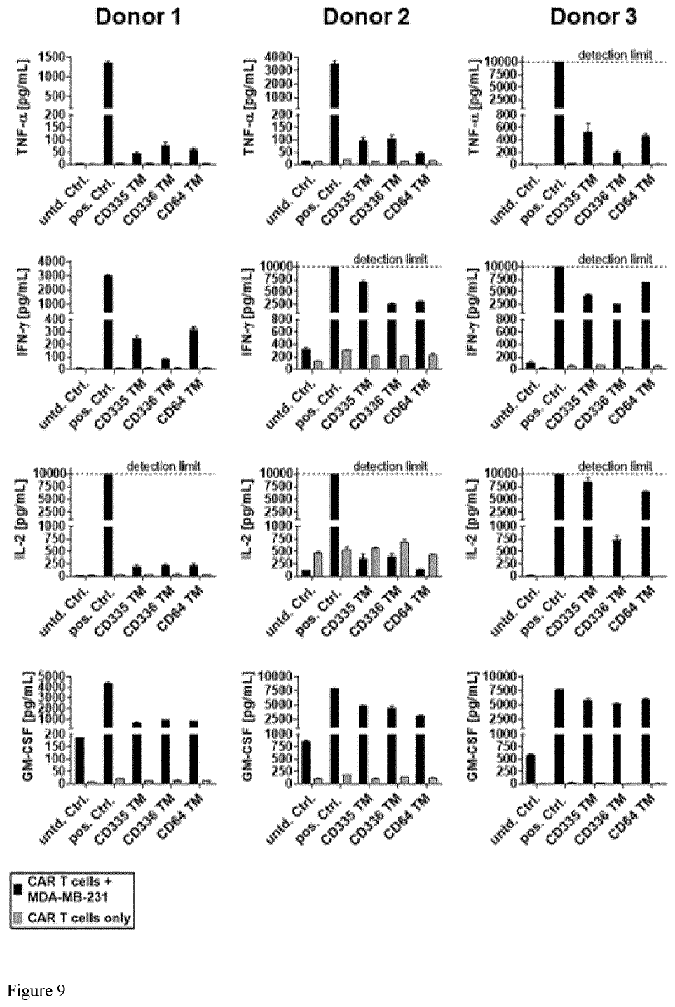

### Example 9: FIG. 9 Shows a Series of Graphs Depicting the Cytokine Expression Profile of Different ESMA-CARs Upon Antigen Stimulation

5*10⁴ CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. To determine background expression of cytokines, CAR+ T cells were cultured without target cells. As a control, unmodified T cells (untd. Ctrl.) were cultured with and without target cells to determine specificity of the CAR-mediated cytokine secretion. After 20 to 24 hours, supernatant was removed and cytokine expression determined by MACSPlex Cytokine 12 Kit, human (Miltenyi Biotec).

After stimulation with target cells, all ESMA-CAR T cells secrete lower level of cytokines than the positive control (pos. Ctrl.) CAR T cells. Secretion levels differ between different donors. TNF-α secretion was over 10 times lower compared to the positive control and IFN-γ secretion was diminished by around 50%. IL-2 was secreted at higher level than background by ESMA-CAR T cells from one donor only (Donor 3), the ESMA-CAR T cells of the other two donors showed IL-2 secretion at level comparable to the background of the untd. Ctrl. CAR T cells cultured without target cells. GM-CSF was secreted by all ESMA-CAR T cells at high level, albeit lower than the pos. Ctrl. CAR T cells. ESMA-CAR T cells and pos. Ctrl. CAR T cells cultured without target cells only showed minimal cytokine expression. The experiment was performed with three donors in triplicate. Mean cytokine expression in pg/mL and standard deviation are shown.

### Example 10: FIG. 10 Illustrates Depicts In Vivo Tumor Burden Control by CD335 ESMA CAR Expressing T Cells in a Xenograft Mouse Model

Tumors were engrafted in NOD scid gamma (NSG) mice by subcutaneous injection of 1×10⁶ TurboRFP-expressing MDA-MB-231 cells in 100 μL PBS in the right flank of mice. Tumor burden was monitored using 2D fluorescence imaging (FLI) with the optical in vivo imaging system (IVIS®) Lumina III (Perkin Elmer) over time. Average fluorescent radiant efficiency of regions of interest (ROIs) was determined to quantify tumor growth. Following seven days of tumor growth, mice were randomized into three groups (n=7 pos. Ctrl & untd. Ctrl., n=6 CD335 ESMA CAR), receiving 2×10⁶ CAR T cells expressing either a positive control CAR (pos. Ctrl.), CD335 ESMA CAR or no CAR (untd Ctrl.) in 100 μL PBS injected into the tail vein. Wellbeing of mice and tumor growth was monitored 2 to 3 times a week over a total duration of 30 days post-CAR T cell injection.

Tumor size within the pos. Ctrl. CAR treated group approached detection limit by day 14, whereas tumors in the untd. Ctrl. continued to grow throughout the whole study. Compared to untd. Ctrl., tumor growth in the CD335 ESMA CAR treated group was significantly diminished from day 17 onward. On the other hand, there were only significant differences on day 10 after T cell injection when comparing pos. Ctrl. and CD335 ESMA CAR treatment groups, illustrating differing kinetics of the ESMA CAR and pos. Ctrl. in diminishing tumor growth. Towards the end of the study, tumor size within the CD335 ESMA CAR treated group was approaching detection limit in all mice except one.

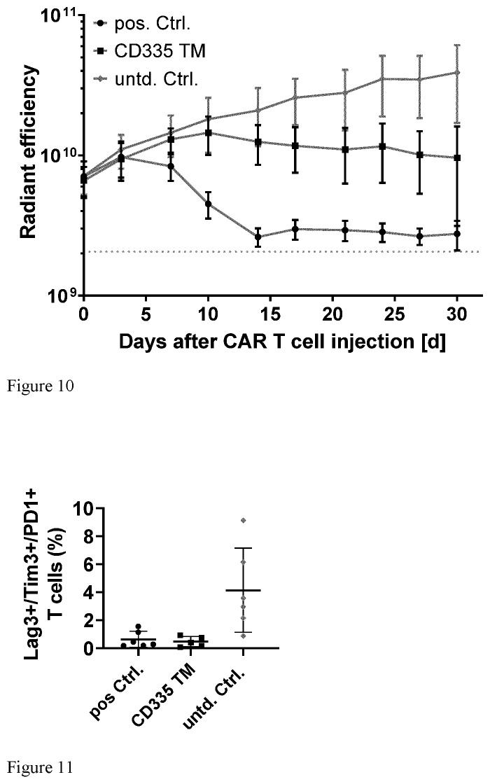

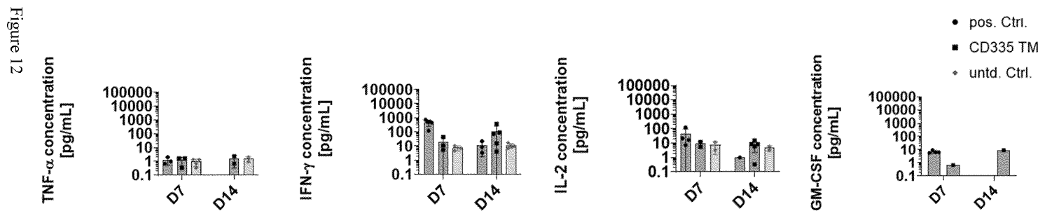

### Example 11: FIG. 11 Depicts Exhaustion Marker Expression on Human T Cells in the Blood of Mice at Endpoint of an In Vivo Study to Analyze Functionality of CD335 ESMA CAR Compared to a Cognate Second Generation CAR

Tumors were engrafted in NOD scid gamma (NSG) mice by subcutaneous injection of 1×10⁶ TurboRFP-expressing MDA-MB-231 cells in 100 μL PBS in the right flank of mice. Following seven days of tumor growth, mice were randomized into three groups (n=7 pos. Ctrl & untd. Ctrl., n=6 CD335 ESMA CAR), receiving 2×10⁶ CAR T cells expressing either the positive 2nd generation CAR control (pos. Ctrl.), CD335 ESMA CAR or no CAR (untd Ctrl.) in 100 μL PBS injected into the tail vein. At study endpoint, 80 μL blood was collected from facial veins of mice and subjected to flow cytometry analysis. T cells were identified by size and granularity (FSC/SSC), live-dead staining with propidium iodide (Miltenyi Biotec), followed by hCD4 or hCD8 expression. Then expression of exhaustion markers was determined with fluorescently labeled antibodies against each marker (Miltenyi Biotec). Mean combined exhaustion marker expression as well as individual values for each mouse and standard deviation are depicted.

When comparing the combined expression of the three exhaustion markers Tim-3, Lag-3 and PD-1 between groups, it could be seen that CD335 ESMA CAR T cells showed a similar expression level to the pos. Ctrl., whereas untd. Ctrl. showed a markedly higher expression of exhaustion markers.

### Example 12: FIG. 12 Shows the Secretion of Human Pro-Inflammatory Cytokines into the Blood of Mice During an In Vivo Study to Analyze Functionality of CD335 ESMA CAR Compared to a Cognate Second Generation CAR

Tumors were engrafted in NOD SCID Gamma (NSG) mice by subcutaneous injection of 1×10⁶ TurboRFP-expressing MDA-MB-231 cells in 100 μL PBS in the right flank of mice. Following seven days of tumor growth, mice were randomized into three groups (n=7 pos. Ctrl & untd. Ctrl., n=6 CD335 ESMA CAR), receiving 2×10⁶ CAR T cells expressing either a positive control CAR (pos. Ctrl.), CD335 ESMA CAR or no CAR (untd Ctrl.) in 100 μL PBS injected into the tail vein. On day 7 and 14 after T cell injection, 80 μL blood was collected from facial veins of mice, then levels of human cytokines in mouse sera were analyzed using the MACSPlex Cytokine 12 Kit, human (Miltenyi Biotec) according to manufacturer's instructions. Mean cytokine expression level as well as individual values for each mouse and standard deviation are depicted.

Substantial and stable IFN-γ and TL-2 cytokine secretion by the CD335 ESMA CAR T cells could be observed on both days. Compared to the pos. Ctrl., cytokine secretion was lower on day 7 but increased on day 14, showing differing kinetics of the CARs. Only minimal levels of TNF-α and GM-CSF close to the detection limit were secreted by each treatment group, no GM-CSF was secreted by the untd. Ctrl.

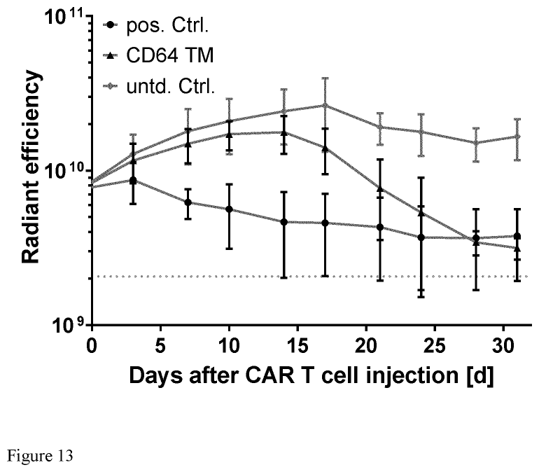

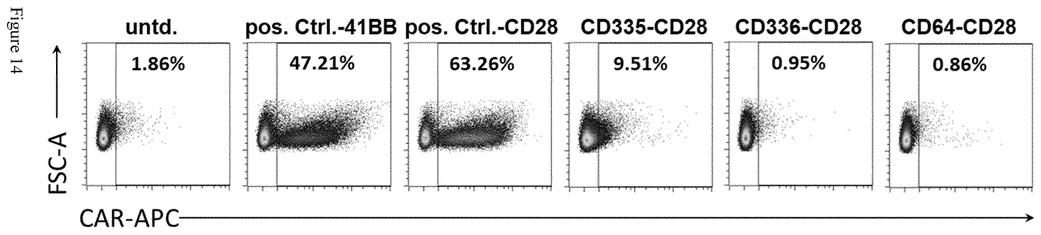

### Example 13: FIG. 13 Displays the Shows In Vivo Tumor Diminishment by CD64 ESMA CAR Expressing T Cells in a Xenograft Mouse Model

Tumors were engrafted in NOD SCID Gamma (NSG) mice by subcutaneous injection of 1×10⁶ TurboRFP-expressing MDA-MB-231 cells in 100 μL PBS in the right flank of mice. Tumor burden was monitored using 2D fluorescence imaging (FLI) with the optical in vivo imaging system (IVIS®) Lumina III (Perkin Elmer) over time. Average fluorescent radiant efficiency of regions of interest (ROIs) was determined to quantify tumor growth. Following seven days of tumor growth, mice were randomized into three groups (n=7), receiving 2×10⁶ CAR T cells expressing either a positive control CAR (pos. Ctrl.), CD64 ESMA CAR or no CAR (untd Ctrl.) in 100 μL PBS injected into the tail vein. Wellbeing of mice and tumor growth was monitored 2 to 3 times a week over a total duration of 31 days post-CAR T cell injection.

Tumor size within the pos. Ctrl. CAR treated group steadily decreased from day 4 onwards and approached detection limit at the end of the study. In the untd. Ctrl., tumors continued growing until day 17, then size stayed stable. In the CD64 ESMA CAR treated group, tumors grew until day 17, then tumor size rapidly decreased, until it approached detection limit by the end of the study. Here, the slower but durable signaling strength of the CD64 ESMA CAR compared to the pos. Ctrl. CAR could be shown.

### Example 14: FIG. 14 Illustrates Shows the Expression of Different CARs with CD28 or 4-IBB Costimulatory Domain on the Cell Surface of Peripheral Blood T Cells

Peripheral blood mononuclear cells (PBMCs) were isolated from buffy coats using gradient centrifugation with Pancoll, human (Pan Biotech). T cells were isolated from PBMCs using the Pan T cell Isolation Kit, human (Miltenyi Biotec). T cells were activated in TexMACS™ medium (Miltenyi Biotec) supplemented with TransAct™ reagent (Miltenyi Biotec), 100 IU/mL of recombinant human IL-2 IS, research grade (Miltenyi Biotec) and 100 μg/mL Primocin® (InvivoGen). Twenty-four hours after isolation, the activated T cells were transduced with CAR-encoding VSV-G pseudo typed lentiviral vectors. Forty-eight hours post transduction, TransAct™ was removed from the cells by medium exchange to TexMACS™ medium supplemented with IL-2 and Primocin®. Cells were expanded for 14 days and CAR expression levels were analyzed on day 14 by flow cytometry using an EGFR protein with His-tag (AcroBiosystems) that is bound by the CAR scFv and detected by secondary anti-His antibody (APC, Miltenyi Biotec). Expression of a positive control CAR with either CD28 (pos. Ctrl.-CD28) or 4-1BB (pos. Ctrl.-41BB) costimulatory domain on the surface of T cells and non-modified T cells (untd. Ctrl.) are shown for comparison. There was only minimal surface expression of the CD335 transmembrane and CD28 costimulatory domain-containing CAR and no detectable expression of the CD336 and CD64 transmembrane domain-containing CARs.

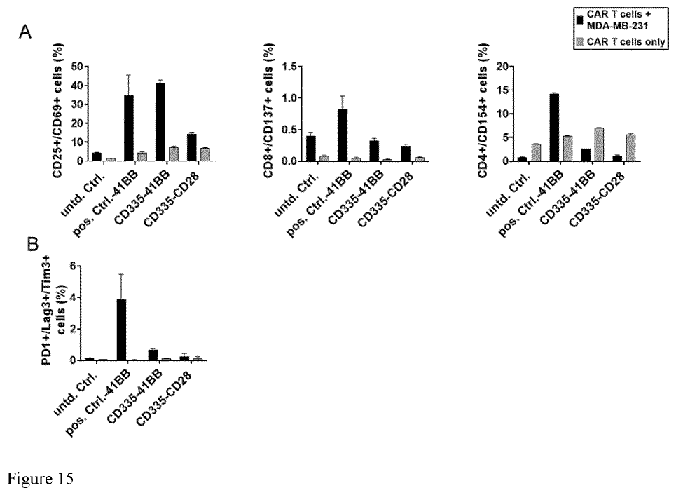

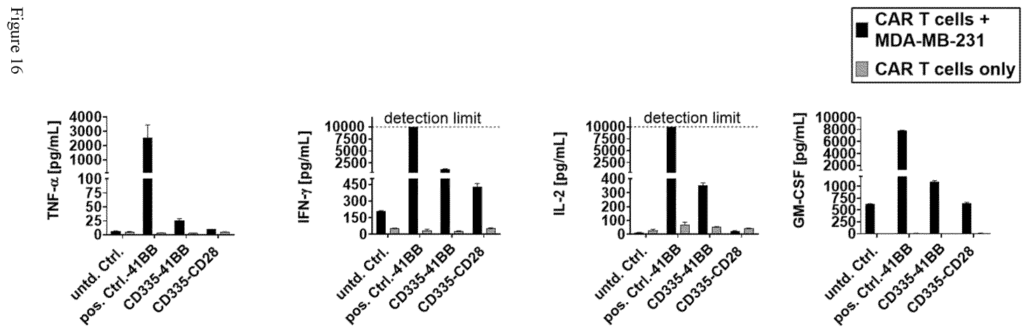

### Example 15: FIG. 15 Depicts the Upregulation of Activation and Exhaustion Markers of CD335 Transmembrane-Encompassing CARs Containing Either a 4-IBB or CD28 Costimulatory Domain

5*10⁴ CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. Then, expression of different activation or exhaustion markers was determined with flow cytometry measurement. Additionally, CAR+ T cells were cultured without target cells to determine background expression of the activation markers. As a control, unmodified T cells (untd. Ctrl.) were cultured with and without target cells to determine baseline marker expression of the T cells. First, CAR+ T cells were identified flow cytometrically, CAR expression was determined with EGFR protein with His-tag and secondary anti-His antibody (APC, Miltenyi Biotec). Then, expression of activation markers (FIG. 15A) and exhaustion markers (FIG. 15B) was determined with fluorescently-labeled antibodies against each marker (Miltenyi Biotec). The experiment was performed in triplicate, mean marker expression and standard deviation are shown.

Upon stimulation with target cells, activation markers CD25, CD69 and CD137 were upregulated in CD335 ESMA CAR T cells (CD335-41BB), whereas only minimal upregulation was visible in the CD28 costimulatory domain-containing cognate CAR (CD335-CD28) (FIG. 15A). There was no upregulation of CD154 in either of the test CARs compared to the background expression in culture without target cells. When comparing combined exhaustion marker expression between the different CARs, only minor upregulation was observed in the CD335 ESMA CAR T cells compared to the positive control CAR (pos. Ctrl.) and no upregulation was visible for the CD28 containing CAR (FIG. 15B), further illustrating that the CD28 containing CAR only shows minimal activation level.

### Example 16: FIG. 16 Depicts the Cytokine Expression Profile of Different CD335 Transmembrane-Encompassing CARs Containing Either a 4-IBB or CD28 Costimulatory Domain

5*10⁴ CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. To determine background expression of cytokines, CAR+ T cells were cultured without target cells. As a control, unmodified T cells (untd. Ctrl.) were cultured with and without target cells to determine baseline cytokine secretion level. After 20 to 24 hours, supernatant was removed and cytokine expression determined by MACSPlex Cytokine 12 Kit, human (Miltenyi Biotec). The experiment was performed in triplicate, mean cytokine expression in pg/mL and standard deviation are shown.

Following stimulation with target cells, all tested CAR T cells secrete cytokines at varying level. Compared to the positive control (pos. Ctrl.) CAR T cells, the 4-1BB containing ESMA CAR and CD28 containing CAR secrete at least 10-fold lower cytokine levels. When comparing the two CARs, it is visible that cytokine secretion of the CD28 containing CAR is strongly diminished, and it does not secrete any IL-2 in contrast to the 4-1BB CAR. Additionally, GM-CSF secretion of the CD28 CAR is close to background level secretion as observed in the untd. Ctrl. No TNF-α was secreted by any of the tested CARs except the pos. Ctrl.

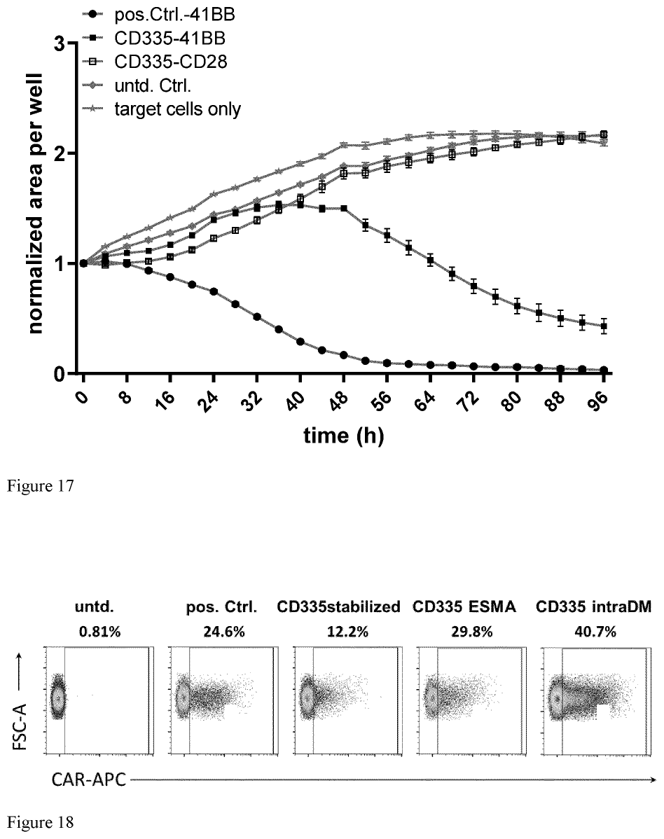

### Example 17: FIG. 17 Displays the Longitudinal Cytotoxicity of Different CD335 Transmembrane-Containing CARs Encompassing Either a 4-1BB or CD28 Costimulatory Domain

5*10⁴ CAR+ T cells were co-cultured with GFP+/Luciferase+ tumor target cells (MDA-MB-231 cells) at 2:1 ratio in DMEM (Biowest) with FBS (Catus Biotech) and Primocin® (InvivoGen) for four days. To determine cytotoxic efficiency of the CAR+ T cells against the tumor cells, the growth of tumor cells was analyzed with an IncuCyte® S3 Life-Cell Analysis System (Sartorius). The green surface area of the target cells was measured over time and values normalized to the start of the experiment (0h). To determine tumor cell growth without T cells, target cells were cultured as a control (target cells only). Target cell growth in co-culture with positive control (pos. Ctrl.) CAR T cells and unmodified T cells (untd. Ctrl.) were measured as a positive and negative control, respectively. The experiment was performed in triplicate, mean target cell area and standard deviation are indicated.

Compared to the pos. Ctrl., there was a timeshift of tumor growth diminishment for the 4-1BB containing CAR, as target cell number started decreasing after 48 hours. In contrast, the CD28 costimulatory domain containing CAR could not diminish tumor growth compared to the untd. Ctrl. and target cell only control.

### Example 18: FIG. 18 Shows the Expression of Different CARs with CD335 Transmembrane and Intracellular Domain as Well as 4-IBB Costimulatory Domain on the Surface of Peripheral Blood T Cells

Surface expression of CARs was determined with flow cytometry using an EGFR protein with His-tag (AcroBiosystems) that is bound by the CAR scFv and detected by secondary anti-His antibody (APC, Miltenyi Biotec). Expression of a CAR containing CD335 transmembrane domain and 4-1BB costimulatory domain (CD335-41BB) is compared to a CAR containing CD335 transmembrane and the cognate intracellular domain in combination with 4-1BB costimulatory domain (CD335stabilized). As controls, a cognate second generation CAR (pos. Ctrl.) and non-modified T cells (untd. Ctrl.) are shown for comparison. While CD335 ESMA CAR is expressed at comparable levels as pos. Ctrl., CD335stabilized is expressed at lower levels and CD335 intraDM at higher levels. Although the CD335 ESMA CAR is expressed at similar levels to the positive control, the CD335stabilized CAR exhibits lower expression levels, while the CD335 intraDM CAR demonstrates higher expression levels.

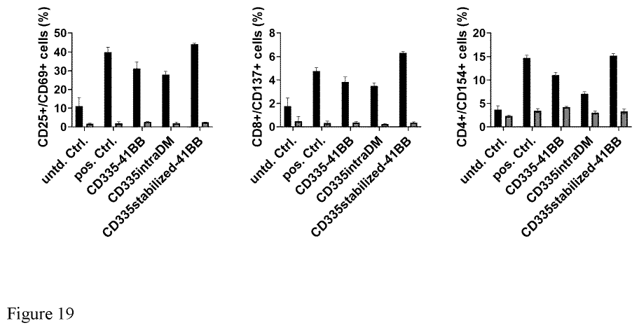

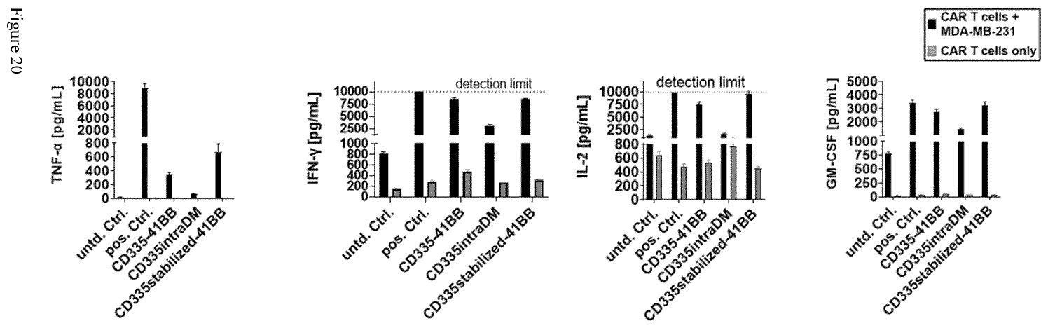

### Example 19: FIG. 19 Depicts the Upregulation of Activation Markers of Different CAR T Cells with CD335 Transmembrane and Intracellular Domain as Well as 4-IBB Costimulatory Domain Upon Co-Culture with Target Cells

5*10⁴ CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. Additionally, CAR+ T cells were cultured without target cells to determine background expression of the activation markers. As a control, unmodified T cells (untd. Ctrl.) were cultured with and without target cells to determine baseline marker expression of the T cells. First, CAR+ T cells were identified flow cytometrically, CAR expression was determined with EGFR protein with His-tag and secondary anti-His antibody (APC, Miltenyi Biotec). Then, expression of activation markers was determined flow cytometrically using fluorescently-labeled antibodies against each marker (Miltenyi Biotec). The experiment was performed in quadruplicate, mean marker expression and standard deviation are shown.

Upon stimulation with target cells, all CARs variants induced the upregulation of activation markers on T cells. Of note, expression of CD25 and CD69 induced by the CD335 stabilized-4-1BB ESMA CAR construct was comparable to the pos. Ctrl, while CD335-4-1BB ESMA and CD335intraDM induced lower levels of CD25 and CD69 upregulation. For CD154 upregulation on CD4+ T cells, a similar potency profile was observed: CD335stabilized-4-1BB ESMA CAR induced CD154 upregulation at similar levels compared to the pos. Ctrl., while CD335-4-1BB ESMA CAR induced slightly lower levels. The lowest efficiency in CD154 upregulation induction was observed with the CD335intraDM construct.

As for the upregulation of CD137 on CD8+ cells, CD335stabilized-4-1BB displayed the highest potency, followed by pos. Ctrl., which was followed by CD335 ESMA CAR and CD335intraDM CAR.

### Example 20: FIG. 20 Illustrates the Cytokine Expression Profile of Different CAR T Cells with CD335 Transmembrane and Intracellular Domain as Well as 4-IBB Costimulatory Domain Upon Co-Culture with Target Cells

5*104 CAR+ T cells were co-cultured with tumor target cells (MDA-MB-231 cells) at 1:2 ratio in DMEM (Biowest) with FBS (Catus Biotech) and Primocin® (InvivoGen) for 20 to 24 hours. To determine background expression of cytokines, CAR+ T cells were cultured without target cells. As a control, unmodified T cells (untd. Ctrl.) were cultured with and without target cells to determine baseline cytokine secretion level. After 20 to 24 hours, supernatant was removed and cytokine expression determined by MACSPlex Cytokine 12 Kit, human (Miltenyi Biotec). The experiment was performed in quadruplicate, mean cytokine expression in pg/mL and standard deviation are shown.

Following stimulation with target cells, all tested CAR T cells secreted cytokines at varying level. For the secretion of IFNγ, IL-2 and TNFα the general hierarchical trend of pos. Ctrl.>CD335stabilized-4-1BB>CD335-4-1BB>CD335intraDM, with pos. Ctrl. displaying the highest potency. For GM-CSF, pos. Ctrl. and CD335stabilized-4-1BB CARs induced comparable levels of cytokine secretion, followed by CD335-4-1BB, which was followed by CD335intraDM.

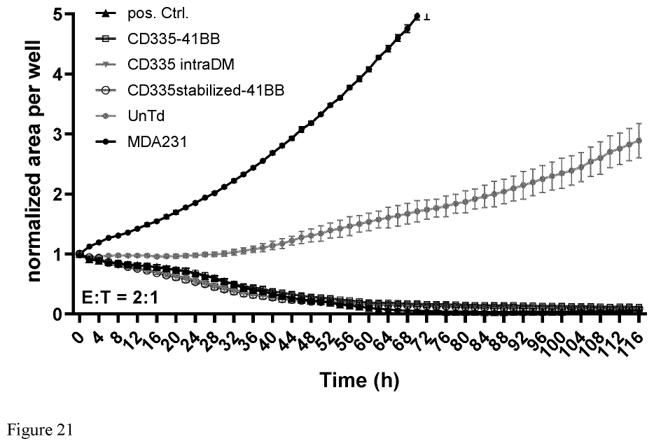

### Example 21: FIG. 21 Displays the Longitudinal Cytotoxicity of Different CAR T Cells with CD335 Transmembrane and Intracellular Domain as Well as 4-IBB Costimulatory Domain

2*10⁴ CAR+ T cells were co-cultured with GFP+/Luciferase+ tumor target cells (MDA-MB-231 cells) at 2:1 ratio in DMEM (Biowest) with FBS (Catus Biotech) and Primocin® (InvivoGen) for four days. To determine cytotoxic efficiency of the CAR+ T cells against the tumor cells, the growth of tumor cells was analyzed with an IncuCyte® S3 Life-Cell Analysis System (Sartorius). The green surface area of the target cells was measured over time and values normalized to the start of the experiment (0h). To determine tumor cell growth without T cells, target cells were cultured as a control (target cells only). Target cell growth in co-culture with positive control (pos. Ctrl.) CAR T cells and unmodified T cells (untd. Ctrl.) were measured as a positive and negative control, respectively. The experiment was performed in quadruplicate, mean target cell area and standard deviation are indicated. All CAR variants were able to eradicate tumor cells at comparable levels to the positive control.

Herein it is referred to the following sequences, e.g. used in the Examples:

(co-stimulatory domain of 4-1BB):
SEQ ID NO: 1
KRGRKKLLYIFKQPFMRPVQTTQEEDGCSCRFPEEEEGGCEL
(co-stimulatory domain of CD28):
SEQ ID NO: 2
RSKRSRLLHSDYMNMTPRRPGPTRKHYQPYAPPRDFAAYRS
(CD8alpha hinge):
SEQ ID NO: 3
TTTPAPRPPTPAPTIASQPLSLRPEACRPAAGGAVHTRGLDFACD
(CD3zeta):
SEQ ID NO: 4
ELQKDKMAEAYSEIGMKGERRRGKGHDGLYQGLSTATKDTYDALHMQALP
PR
(transmembrane domain of CD8alpha):
SEQ ID NO: 5
IYIWAPLAGTCGVLLLSLVITLYC
(reference CAR):
SEQ ID NO: 6
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR
(transmembrane domain of CD64):
SEQ ID NO: 7
FHVLFYLAVGIMFLVNTVLWVTI
(transmembrane domain of CD335):
SEQ ID NO: 8
LLRMGLAFLVLVALVWFLV
(transmembrane domain of CD336):
SEQ ID NO: 9
LVPVFCGLLVAKSLVLSALLV
(transmembrane domain of CD337):
SEQ ID NO: 10
AGTVLLLRAGFYAVSFLSVAV
(transmembrane domain of CD16):
SEQ ID NO: 11
VSFCLVMVLLFAVDTGLYFSV
(transmembrane domain of NKG2D):
SEQ ID NO: 12
AVMIIFRIGMAVAIFCCFFFP
(transmembrane domain of NKG2C):
SEQ ID NO: 13
LVITKLVTAMLVICIIGLVEATL
(transmembrane domain of KIR2DS2):
SEQ ID NO: 14
VLIGTSVVKIPFTILLFFLL
(EGFR scFv):
SEQ ID NO: 15
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSS
(MSLN scFv):
SEQ ID NO: 16
EVQLVQSGGGLVQPGGSLRLSCAASGFTFDDYAMHWVRQAPGKGLEWVSG
ISWNSGSIGYADSVKGRFTISRDNAKNSLYLQMNSLRAEDTALYYCAKDL
SSVAGPFNYWGQGTLVTVSSGGGGSGGGGSGGGGSSSELTQDPAVSVALG
QTVRITCQGDSLRSYYASWYQQKPGQAPVLVIYGKNNRPSGIPDRFSGSS
SGNTASLTITGAQAEDEADYYCNSRDSSGNHLVFGGGTQLTVLG
(CD318 scFv):
SEQ ID NO: 17
EVQLQQSGAELVRPGALVKLSCKASGFNIKDYYIHWVKQRPEQGLEWIGW
IDPENGHTIYDPKFQGKASITADTSSNTAYLQLSSLTSEDTAVYYCARLT
GTTYAMDYWGQGTSVTVSSGGGGSGGGGSGGGGSDIVMTQSHKFMSTSVG
DRVSITCKASQDVSTAVAWYQQKSGQSPKLLIYWASTRHTGVPDRFTGSG
SGTDYTLTISSVQAEDLALYYCQQHYSTPYTFGGGTKLEIK
(CD335 TM CAR w/o scFv):
SEQ ID NO: 18
TTTPAPRPPTPAPTIASQPLSLRPEACRPAAGGAVHTRGLDFACDLLRMG
LAFLVLVALVWFLVKRGRKKLLYIFKQPFMRPVQTTQEEDGCSCRFPEEE
EGGCEL
(CD336 TM CAR w/o scFv):
SEQ ID NO: 19
TTTPAPRPPTPAPTIASQPLSLRPEACRPAAGGAVHTRGLDFACDLVPVF
CGLLVAKSLVLSALLVKRGRKKLLYIFKQPFMRPVQTTQEEDGCSCRFPE
EEEGGCEL
(CD64 TM CAR w/o scFv):
SEQ ID NO: 20
TTTPAPRPPTPAPTIASQPLSLRPEACRPAAGGAVHTRGLDFACDFHVLF
YLAVGIMFLVNTVLWVTIKRGRKKLLYIFKQPFMRPVQTTQEEDGCSCRF
PEEEEGGCEL
(ROR1 scFv):
SEQ ID NO: 21
QAAQVQLQQSGAEVKKPGSSVKVSCKASGGTFSSYAISWVRQAPGQGLEW
MGWINPNSGGTNYAQRFQGRVTMTRDTSISTAYMELSRLRSDDTAVYYCA
SYNDAFDIWGQGTLVTVSSGGGGSGGGGSGGGGSNFMLTQPHSVSESPGK
TVTISCTRSSGSIASNYVQWYQQRPGSAPTIVIYEDDQRPSGVPDRFSGS
IDTSSNSASLTISGLQSEDEADYYCQSYEPGNGVFGGGTKVTVL
(EGFR Nanobody):
SEQ ID NO: 22
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSS
(CD335 TM CAR w/ EGFR scFv):
SEQ ID NO: 23
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDLLRMGLAFLV
LVALVWFLVKRGRKKLLYIFKQPFMRPVQTTQEEDGCSCRFPEEEEGGCE
L
(CD336 TM CAR w/EGFR scFv):
SEQ ID NO: 24
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDLVPVFCGLLV
AKSLVLSALLVKRGRKKLLYIFKQPFMRPVQTTQEEDGCSCRFPEEEEGG
CEL
(CD64 TM CAR w/EGFR scFv):
SEQ ID NO: 25
QILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDFHVLFYLAVG
IMFLVNTVLWVTIKRGRKKLLYIFKQPFMRPVQTTQEEDGCSCRFPEEEE
GGCEL
(CD16 TM CAR w/ EGFR scFv):
SEQ ID NO: 26
QILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDVSFCLVMVLL
FAVDTGLYFSVKRGRKKLLYIFKQPFMRPVQTTQEEDGCSCRFPEEEEGG
CEL
(CD337 TM CAR w/ EGFR scFv):
SEQ ID NO: 27
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDAGTVLLLRAG
FYAVSFLSVAVKRGRKKLLYIFKQPFMRPVQTTQEEDGCSCRFPEEEEGG
CEL
(NKG2D TM CAR w/ EGFR scFv):
SEQ ID NO: 28
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDAVMIIFRIGM
AVAIFCCFFFPKRGRKKLLYIFKQPFMRPVQTTQEEDGCSCRFPEEEEGG
CEL
(NKG2C TM CAR w/ EGFR scFv):
SEQ ID NO: 29
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDLVITKLVTAM
LVICIIGLVEATLKRGRKKLLYIFKQPFMRPVQTTQEEDGCSCRFPEEEE
GGCEL
(KIR2DS2 TM CAR w/ EGFR scFv):
SEQ ID NO: 30
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDVLIGTSVVKI
PFTILLFFLLKRGRKKLLYIFKQPFMRPVQTTQEEDGCSCRFPEEEEGGC
EL
(CD335 intracellular domain):
SEQ ID NO: 31
EDWLSRKRTRERASRASTWEGRRRLNTQTL
(CD335 transmembrane and intracellular
domain):
SEQ ID NO: 32
LLRMGLAFLVLVALVWFLVEDWLSRKRTRERASRASTWEGRRRLNTQTL
(CD335 intraDM w/o scFv):
SEQ ID NO: 33
TTTPAPRPPTPAPTIASQPLSLRPEACRPAAGGAVHTRGLDFACDLLRMG
LAFLVLVALVWFLVEDWLSRKRTRERASRASTWEGRRRLNTQTL
(CD335 intraDM CAR w/EGFR scFv):
SEQ ID NO: 34
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDLLRMGLAFLV
LVALVWFLVEDWLSRKRTRERASRASTWEGRRRLNTQTL
(CD335 stabilized 4-1BB CAR w/o scFv):
SEQ ID NO: 35
TTTPAPRPPTPAPTIASQPLSLRPEACRPAAGGAVHTRGLDFACDLLRMG
LAFLVLVALVWFLVEDWLSRKRTRERASRASTWEGRRRLNTQTLKRGRKK
LLYIFKQPFMRPVQTTQEEDGCSCRFPEEEEGGCEL
(CD335 stabilized 4-1BB CAR w/ EGFR
scFv):
SEQ ID NO: 36
QILLTQSPVILSVSPGERVSFSCRASQSIGTNIHWYQQRINGSPRLLIKY
ASESISGIPSRFSGSGSGTDFTLSINSVESEDIADYYCQQNNNWPTTFGA
GTKLELKRGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSAAATTTPA
PRPPTPAPTIASQPLSLRPEACRPAAGGAVHTRGLDFACDLLRMGLAFLV
LVALVWFLVEDWLSRKRTRERASRASTWEGRRRLNTQTLKRGRKKLLYIF
KQPFMRPVQTTQEEDGCSCRFPEEEEGGCEL
(CD336 intracellular domain):
SEQ ID NO: 37
WWGDIWWKTMMELRSLDTQKATCHLQQVTDLPWTSVSSPVEREILYHTVA
RTKISDDDDEHTL
(CD336 transmembrane and intracellular
domain):
SEQ ID NO: 38
LVPVFCGLLVAKSLVLSALLVWWGDIWWKTMMELRSLDTQKATCHLQQVT
DLPWTSVSSPVEREILYHTVARTKISDDDDEHTL
(CD336 stabilized 4-1BB CAR w/o
scFv):
SEQ ID NO: 39
TTTPAPRPPTPAPTIASQPLSLRPEACRPAAGGAVHTRGLDFACDLVPVF
CGLLVAKSLVLSALLVWWGDIWWKTMMELRSLDTQKATCHLQQVTDLPWT
SVSSPVEREILYHTVARTKISDDDDEHTLKRGRKKLLYIFKQPFMRPVQT
TQEEDGCSCRFPEEEEGGCEL
(CD64 intracellular domain):
SEQ ID NO: 40
RKELKRKKKWDLEISLDSGHEKKVISSLQEDRHLEEELKCQEQKEEQLQE
GVHRKEPQGAT
(CD64 transmembrane and intracellular
domain):
SEQ ID NO: 41
VLFYLAVGIMFLVNTVLWVTIRKELKRKKKWDLEISLDSGHEKKVISSLQ
EDRHLEEELKCQEQKEEQLQEGVHRKEPQGAT
(CD64 stabilized 4-1BB CAR w/o
scFv):
SEQ ID NO: 42
TTTPAPRPPTPAPTIASQPLSLRPEACRPAAGGAVHTRGLDFACDVLFYL
AVGIMFLVNTVLWVTIRKELKRKKKWDLEISLDSGHEKKVISSLQEDRHL
EEELKCQEQKEEQLQEGVHRKEPQGATKRGRKKLLYIFKQPFMRPVQTTQ
EEDGCSCRFPEEEEGGCEL

The invention claimed is:

1. An endogenous signaling molecule activating chimeric antigen receptor (ESMA-CAR) comprising
a) an antigen binding domain specific for an antigen
b) a first transmembrane domain
c) an intracellular signaling domain comprising a co-stimulatory domain but no stimulatory domain, wherein said first transmembrane domain,
when expressed on the cell surface of an immune cell, is able to recruit a stimulatory domain of an endogenous signaling molecule of said immune cell, wherein said endogenous signaling molecule is a protein comprising a second transmembrane domain and an intracellular signaling domain comprising a stimulatory domain, and wherein the interaction of the first transmembrane domain and the second transmembrane domain activates said immune cell upon binding of said antigen to said antigen binding domain of said ESMA-CAR.

2. The ESMA-CAR according to claim 1, wherein said first transmembrane domain recruits a stimulatory domain of an endogenous signaling molecule that comprises CD3gamma, CD3delta, CD3epsilon, CD3zeta CD3zeta, DAP10, DAP12 or FcRgamma.

3. The ESMA-CAR according to claim 1, wherein said ESMA-CAR expressed in said immune cell displays at least 40% cytotoxicity against target cells that express said antigen as compared to a reference CAR expressed in said immune cell and wherein said ESMA-CAR expressed in said immune cell displays upon antigen stimulation an at least 3-fold lower cytokine secretion of at least one cytokine as compared to said reference CAR expressed in said immune cell, wherein said at least one cytokine is selected from the group of IL-2, ZFN-y, TNF-a and GM-CSF, and wherein said reference CAR comprises
a) an antigen binding domain, wherein said antigen binding domain is identical to the antigen binding domain of said ESMA-CAR;
b) a transmembrane domain that is the transmembrane domain of CD8alpha; and
c) an intracellular signaling domain comprising a co-stimulatory domain and a stimulatory domain comprising CD3zeta, wherein said co-stimulatory domain is identical to the co-stimulatory domain of said ESMA-CAR.

4. The ESMA-CAR according to claim 3, wherein said ESMA-CAR expressed in said immune cell additionally displays
i) an upregulation of at least one activation marker selected from the group consisting of activation markers CD69, CD25, CD137, and CD154, wherein said upregulation of said at least one activation marker is at least 5-fold as compared to said immune cell that is not genetically engineered to express a CAR and/or
ii) an at least 25% lower upregulation of at least one exhaustion marker as compared to said reference CAR expressed in said immune cell, wherein said at least one exhausting marker is selected from the exhausting markers LAG3, TIM3 and PD-1.

5. The ESMA-CAR according to claim 1, wherein said first transmembrane domain is the transmembrane domain of CD64, CD335 or CD336.

6. (canceled)

7. An immune cell expressing an ESMA-CAR according to claim 1.

8. The immune cell according to claim 7, wherein said immune cell is a T cell or an NK cell.

9. The immune cell according to claim 7 for use in treatment of a disease.

10. An in vitro method for the generation of an endogenous signaling molecule activating chimeric antigen receptor (ESMA-CAR) comprising
i) an antigen binding domain specific for an antigen
ii) a first transmembrane domain
iii) an intracellular signaling domain comprising a costimulatory domain but no stimulatory domain,
the method comprising the step of selecting an ESMA-CAR that has a first transmembrane domain that, when expressed on the cell surface of an immune cell, is able to recruit a stimulatory domain of an endogenous signaling molecule of said immune cell,
wherein said endogenous signaling molecule is a protein comprising a second transmembrane domain and an intracellular signaling domain comprising a stimulatory domain, and
wherein the interaction of the first transmembrane domain and the second transmembrane domain activates said immune cell upon binding of said antigen to said antigen binding domain of said ESMA-CAR.

11. The method according to claim 10, wherein said selecting of an ESMA-CAR comprises the steps of
a) selecting an ESMA-CAR with a first transmembrane domain that can be expressed on the cell surface of an immune cell, and subsequently
b) selecting an ESMA-CAR with said first transmembrane domain that can activate said immune cell upon binding of said antigen to said antigen binding domain of said CAR.

12. The method according to claim 11, wherein said method comprises identifying an ESMA-CAR of step b) that, when expressed in said immune cell displays at least 40% cytotoxicity against target cells that express said antigen as compared to a reference CAR expressed in said immune cell and wherein said ESMA-CAR expressed in said immune cell displays upon antigen stimulation an at least 3-fold lower cytokine secretion of at least one cytokine as compared to said reference CAR expressed in said immune cell,
wherein the at least one cytokine is selected from the group of IL-2, IFN-y, TNF-a and GM-CSF, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta, wherein said co-stimulatory domain is identical to the costimulatory domain of said ESMA-CAR.

13. The method according to claim 12, wherein said identifying an ESMA-CAR of step b) that, when expressed in said immune cell additionally displays
i) an upregulation of at least one activation marker selected from the group consisting of activation markers CD69, CD25, CD137, and CD154, wherein said upregulation of said at least one activation marker is at least 5-fold as compared to said immune cell that is not genetically engineered to express a CAR and/or
ii) an at least 25% lower upregulation of at least one exhaustion marker as compared to said reference CAR expressed in said immune cell, wherein said at least one exhausting marker is selected from the exhausting markers LAG3, TIM3 and PD-1.

14. The method according to claim 10, wherein said immune cell is a T cell or an NK cell.

15. An in vitro method for assessing the efficiency of an ESMA-CAR, the method comprising the steps
a) introducing a nucleic acid encoding an ESMA-CAR into an immune cell, said ESMA-CAR comprising
i) an antigen binding domain specific for an antigen
ii) a first transmembrane domain
iii) an intracellular signaling domain comprising a costimulatory domain but no stimulatory domain,
wherein said first transmembrane domain, when expressed on the cell surface of said immune cell, is able to recruit a stimulatory domain of an endogenous signaling molecule of said immune cell,
wherein said endogenous signaling molecule is a protein comprising a second transmembrane domain and an intracellular signaling domain comprising a stimulatory domain, and
wherein the interaction of the first transmembrane domain and the second transmembrane domain activates said immune cell upon binding of said antigen to said antigen binding domain of said ESMA-CAR,
b) determining if said ESMA-CAR, when expressed in said immune cell, displays at least 40% cytotoxicity against target cells that express said antigen as compared to a reference CAR expressed in said immune cell and wherein said ESMA-CAR expressed in said immune cell displays upon antigen stimulation an at least 3-fold lower cytokine secretion of at least one cytokine as compared to said reference CAR expressed in said immune cell,
wherein the at least one cytokine is selected from the group of IL-2, IFN-y, TNF-a and GM-CSF, and wherein said reference CAR comprises
a) an antigen binding domain, wherein said antigen binding domain is identical to the antigen binding domain of said ESMA-CAR
b) a transmembrane domain that is the transmembrane domain of CD8alpha
c) an intracellular signaling domain comprising a co-stimulatory domain and a stimulatory domain comprising CD3zeta,
wherein said co-stimulatory domain is identical to the costimulatory domain of said ESMA-CAR, and wherein said at least 40% cytotoxicity and said at least 3-fold lower cytokine secretion of at least one cytokine is indicative for a good efficiency of said ESMA-CAR in said immune cell.

16. An endogenous signaling molecule activating chimeric antigen receptor (ESMA-CAR) obtained according to the method of claim 10.


SEQUENCE LISTING (42 sequences)

SEQ ID NO: 1 - protein, 42 aa, synthetic construct
KRGRKKLLYIFKQPFMRPVQTTQEEDGCSCRFPEEEEGGCEL

SEQ ID NO: 2 - protein, 41 aa, synthetic construct
RSKRSRLLHSDYMNMTPRRPGPTRKHYQPYAPPRDFAAYRS

SEQ ID NO: 3 - protein, 45 aa, synthetic construct
TTTPAPRPPTPAPTIASQPLSLRPEACRPAAGGAVHTRGLDFACD

SEQ ID NO: 4 - protein, 52 aa, synthetic construct
ELQKDKMAEAYSEIGMKGERRRGKGHDGLYQGLSTATKDTYDALHMQALPPR

SEQ ID NO: 5 - protein, 24 aa, synthetic construct
IYIWAPLAGTCGVLLLSLVITLYC

SEQ ID NO: 6 - protein, 468 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 23 aa, synthetic construct
FHVLFYLAVGIMFLVNTVLWVTI

SEQ ID NO: 8 - protein, 19 aa, synthetic construct
LLRMGLAFLVLVALVWFLV

SEQ ID NO: 9 - protein, 21 aa, synthetic construct
LVPVFCGLLVAKSLVLSALLV

SEQ ID NO: 10 - protein, 21 aa, synthetic construct
AGTVLLLRAGFYAVSFLSVAV

SEQ ID NO: 11 - protein, 21 aa, synthetic construct
VSFCLVMVLLFAVDTGLYFSV

SEQ ID NO: 12 - protein, 21 aa, synthetic construct
AVMIIFRIGMAVAIFCCFFFP

SEQ ID NO: 13 - protein, 23 aa, synthetic construct
LVITKLVTAMLVICIIGLVEATL

SEQ ID NO: 14 - protein, 20 aa, synthetic construct
VLIGTSVVKIPFTILLFFLL

SEQ ID NO: 15 - protein, 242 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 16 - protein, 244 aa, synthetic construct
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLG

SEQ ID NO: 17 - protein, 241 aa, synthetic construct
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSSGGGGSGGGGSGGGGSDIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 18 - protein, 106 aa, synthetic construct
TTTPAPRPPTPAPTIASQPLSLRPEACRPAAGGAVHTRGLDFACDLLRMGLAFLVLVALVWFLVKRGRKKLLYIFKQPFMRPVQTTQEEDGCSCRFPEEEEGGCEL

SEQ ID NO: 19 - protein, 108 aa, synthetic construct
TTTPAPRPPTPAPTIASQPLSLRPEACRPAAGGAVHTRGLDFACDLVPVFCGLLVAKSLVLSALLVKRGRKKLLYIFKQPFMRPVQTTQEEDGCSCRFPEEEEGGCEL

SEQ ID NO: 20 - protein, 110 aa, synthetic construct
TTTPAPRPPTPAPTIASQPLSLRPEACRPAAGGAVHTRGLDFACDFHVLFYLAVGIMFLVNTVLWVTIKRGRKKLLYIFKQPFMRPVQTTQEEDGCSCRFPEEEEGGCEL

SEQ ID NO: 21 - protein, 244 aa, synthetic construct
QAAQVQLQQSGAEVKKPGSSVKVSCKASGGTFSSYAISWVRQAPGQGLEWMGWINPNSGGTNYAQRFQGRVTMTRDTSISTAYMELSRLRSDDTAVYYCASYNDAFDIWGQGTLVTVSSGGGGSGGGGSGGGGSNFMLTQPHSVSESPGKTVTISCTRSSGSIASNYVQWYQQRPGSAPTIVIYEDDQRPSGVPDRFSGSIDTSSNSASLTISGLQSEDEADYYCQSYEPGNGVFGGGTKVTVL

SEQ ID NO: 22 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 23 - protein, 351 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDLLRMGLAFLVLVALVWFLVKRGRKKLLYIFKQPFMRPVQTTQEEDGCSCRFPEEEEGGCEL

SEQ ID NO: 24 - protein, 353 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDLVPVFCGLLVAKSLVLSALLVKRGRKKLLYIFKQPFMRPVQTTQEEDGCSCRFPEEEEGGCEL

SEQ ID NO: 25 - protein, 355 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDFHVLFYLAVGIMFLVNTVLWVTIKRGRKKLLYIFKQPFMRPVQTTQEEDGCSCRFPEEEEGGCEL

SEQ ID NO: 26 - protein, 353 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDVSFCLVMVLLFAVDTGLYFSVKRGRKKLLYIFKQPFMRPVQTTQEEDGCSCRFPEEEEGGCEL

SEQ ID NO: 27 - protein, 353 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDAGTVLLLRAGFYAVSFLSVAVKRGRKKLLYIFKQPFMRPVQTTQEEDGCSCRFPEEEEGGCEL

SEQ ID NO: 28 - protein, 353 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDAVMIIFRIGMAVAIFCCFFFPKRGRKKLLYIFKQPFMRPVQTTQEEDGCSCRFPEEEEGGCEL

SEQ ID NO: 29 - protein, 355 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDLVITKLVTAMLVICIIGLVEATLKRGRKKLLYIFKQPFMRPVQTTQEEDGCSCRFPEEEEGGCEL

SEQ ID NO: 30 - protein, 352 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDVLIGTSVVKIPFTILLFFLLKRGRKKLLYIFKQPFMRPVQTTQEEDGCSCRFPEEEEGGCEL

SEQ ID NO: 31 - protein, 30 aa, synthetic construct
EDWLSRKRTRERASRASTWEGRRRLNTQTL

SEQ ID NO: 32 - protein, 49 aa, synthetic construct
LLRMGLAFLVLVALVWFLVEDWLSRKRTRERASRASTWEGRRRLNTQTL

SEQ ID NO: 33 - protein, 94 aa, synthetic construct
TTTPAPRPPTPAPTIASQPLSLRPEACRPAAGGAVHTRGLDFACDLLRMGLAFLVLVALVWFLVEDWLSRKRTRERASRASTWEGRRRLNTQTL

SEQ ID NO: 34 - protein, 339 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDLLRMGLAFLVLVALVWFLVEDWLSRKRTRERASRASTWEGRRRLNTQTL

SEQ ID NO: 35 - protein, 136 aa, synthetic construct
TTTPAPRPPTPAPTIASQPLSLRPEACRPAAGGAVHTRGLDFACDLLRMGLAFLVLVALVWFLVEDWLSRKRTRERASRASTWEGRRRLNTQTLKRGRKKLLYIFKQPFMRPVQTTQEEDGCSCRFPEEEEGGCEL

SEQ ID NO: 36 - protein, 381 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSAAATTTPAPRPPTPAPTIASQPLSLRPEACRPAAGGAVHTRGLDFACDLLRMGLAFLVLVALVWFLVEDWLSRKRTRERASRASTWEGRRRLNTQTLKRGRKKLLYIFKQPFMRPVQTTQEEDGCSCRFPEEEEGGCEL

SEQ ID NO: 37 - protein, 63 aa, synthetic construct
WWGDIWWKTMMELRSLDTQKATCHLQQVTDLPWTSVSSPVEREILYHTVARTKISDDDDEHTL

SEQ ID NO: 38 - protein, 84 aa, synthetic construct
LVPVFCGLLVAKSLVLSALLVWWGDIWWKTMMELRSLDTQKATCHLQQVTDLPWTSVSSPVEREILYHTVARTKISDDDDEHTL

SEQ ID NO: 39 - protein, 171 aa, synthetic construct
TTTPAPRPPTPAPTIASQPLSLRPEACRPAAGGAVHTRGLDFACDLVPVFCGLLVAKSLVLSALLVWWGDIWWKTMMELRSLDTQKATCHLQQVTDLPWTSVSSPVEREILYHTVARTKISDDDDEHTLKRGRKKLLYIFKQPFMRPVQTTQEEDGCSCRFPEEEEGGCEL

SEQ ID NO: 40 - protein, 61 aa, synthetic construct
RKELKRKKKWDLEISLDSGHEKKVISSLQEDRHLEEELKCQEQKEEQLQEGVHRKEPQGAT

SEQ ID NO: 41 - protein, 82 aa, synthetic construct
VLFYLAVGIMFLVNTVLWVTIRKELKRKKKWDLEISLDSGHEKKVISSLQEDRHLEEELKCQEQKEEQLQEGVHRKEPQGAT

SEQ ID NO: 42 - protein, 169 aa, synthetic construct
TTTPAPRPPTPAPTIASQPLSLRPEACRPAAGGAVHTRGLDFACDVLFYLAVGIMFLVNTVLWVTIRKELKRKKKWDLEISLDSGHEKKVISSLQEDRHLEEELKCQEQKEEQLQEGVHRKEPQGATKRGRKKLLYIFKQPFMRPVQTTQEEDGCSCRFPEEEEGGCEL